(19) United States
(12) Patent Application Publication
(10) Pub. No.: US 2007/0118089 A1
(43) Pub. Date: May 24, 2007

(54) Disposable absorbent article having backsheet strips

(75) Inventor: Gary Dean LaVon, Liberty Township, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/286,614

(22) Filed: Nov. 23, 2005

(51) Int. Cl. A61F 13/15

(52) U.S. Cl. 604/385.28; 604/385.22

(57) ABSTRACT

A disposable pull-on absorbent article includes two laterally opposing longitudinally extending backsheet strips attached to an exterior surface of an absorbent assembly in laterally opposing attachment zones. Each backsheet strip may include a water-impermeable layer and may be extensible. The absorbent assembly includes laterally opposing side flaps which may be formed by folding portions of the absorbent assembly laterally inward. A longitudinally extending elastic gathering member is attached to each side flap adjacent to its proximal edge. When the article is worn, the elastic gathering members contract and raise the side flaps to form side barriers. The absorbent assembly includes an absorbent core that may contain superabsorbent particles, which may be contained inside pockets. A portion of the absorbent assembly such as the portion that lies between the backsheet strip attachment zones may be extensible and may include a water-impermeable layer.

### FIELD OF THE INVENTION

This invention relates to disposable absorbent articles such as disposable diapers and other articles intended for use on incontinent persons.

### BACKGROUND OF THE INVENTION

Disposable absorbent articles are designed to absorb and contain bodily waste in order to prevent soiling of the body and clothing of the wearer, as well as bedding or other objects with which the wearer comes into contact. Pant-like garments, especially those of the “pull-on” type, include a pair of closed side interfaces that predefine encircled waist and leg openings. Accordingly, pull-on diapers can be more easily applied especially to a standing wearer than taped diapers, which require manual fastening to secure the diaper on the wearer.

As the usage of disposable absorbent articles has expanded, their complexity has increased with the incorporation of additional features serving to enhance their performance and appearance. The costs of the materials and the costs of the manufacturing processes have also increased in conjunction with the increase in complexity. As a result, the prices at which these articles are sold have risen to levels that many potential purchasers around the world cannot afford to pay. Thus, a need exists for a simple disposable absorbent article.

### SUMMARY OF THE INVENTION

In accordance with one aspect of the present invention, a disposable pant-like garment is provided having a front waist region, a back waist region, and a crotch region disposed between the waist regions. The garment includes laterally opposing first and second side edge regions. Each side edge region includes a pre-closed side interface. The pre-closed side interfaces define an encircled waist opening and a pair of encircled leg openings. The garment further includes an absorbent assembly that has an interior surface, an exterior surface, and laterally opposing longitudinally extending side flaps. Each side flap has longitudinally opposing ends and a longitudinally extending proximal edge. Each side flap is attached adjacent to its ends to the interior surface of the absorbent assembly. Each side flap also has a longitudinally extending elastic gathering member attached adjacent to its proximal edge. When allowed to relax the elastic gathering member contracts and lifts the proximal edge away from the interior surface of the absorbent assembly, thereby raising the side flap to form a side barrier. The garment further includes a pair of laterally opposing longitudinally extending backsheet strips attached to the exterior surface of the absorbent assembly. At least a portion of the absorbent assembly is laterally extensible.

In accordance with another aspect of the present invention, a disposable pant-like garment has a front waist region, a back waist region, and a crotch region disposed between the waist regions. The garment includes laterally opposing first and second side edge regions. Each side edge region includes a pre-closed side interface. The pre-closed side interfaces define an encircled waist opening and a pair of encircled leg openings. The garment further includes an absorbent assembly having an interior surface, an exterior surface, and laterally opposing longitudinally extending side flaps. Each side flap has longitudinally opposing ends and a longitudinally extending proximal edge. Each side flap is attached adjacent to its ends to the interior surface of the absorbent assembly. Each side flap has a longitudinally extending elastic gathering member attached adjacent to its proximal edge. When allowed to relax the elastic gathering member contracts and lifts the proximal edge away from the interior surface of the absorbent assembly, thereby raising the side flap to form a side barrier. The garment further includes a pair of laterally opposing longitudinally extending backsheet strips attached to the exterior surface of the absorbent assembly. At least a portion of at least one of the backsheet strips is laterally extensible.

In accordance with yet another aspect of the invention, a disposable pant-like garment has a front waist region, a back waist region, and a crotch region disposed between the waist regions. The garment includes laterally opposing first and second side edge regions. Each side edge region includes a pre-closed side interface. The pre-closed side interfaces define an encircled waist opening and a pair of encircled leg openings. The garment further includes an absorbent assembly having an interior surface, an exterior surface, and laterally opposing longitudinally extending side flaps. Each side flap has longitudinally opposing ends and a longitudinally extending proximal edge. Each side flap is attached adjacent to its ends to the interior surface of the absorbent assembly. Each side flap has a longitudinally extending elastic gathering member attached adjacent to its proximal edge. When allowed to relax the elastic gathering member contracts and lifts the proximal edge away from the interior surface of the absorbent assembly, thereby raising the side flap to form a side barrier. The garment further includes a pair of laterally opposing longitudinally extending backsheet strips attached to the exterior surface of the absorbent assembly. The absorbent assembly includes an absorbent core storage component containing no airfelt.

### BRIEF DESCRIPTION OF THE DRAWINGS

In the accompanying drawing figures, like reference numerals identify like elements, which may or may not be identical in the several exemplary embodiments that are depicted. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description.

FIG. 1 is a plan view of an exemplary disposable absorbent article shown in its flat, uncontracted state (i.e., without the contraction induced by elastic members) prior to being formed into a pull-on pant, wherein the interior portion of the absorbent article that faces inwardly toward the wearer and contacts the wearer is shown facing the viewer;

FIG. 2 is a plan view of the absorbent article illustrated in FIG. 1 in its flat, uncontracted state prior to being formed into a pull-on pant, with the exterior portion of the article that faces outwardly away from the wearer shown facing the viewer;

FIG. 3 is a section view of the absorbent article illustrated in FIGS. 1 and 2 taken along line 3-3 prior to being formed into a pull-on pant, wherein the interior portion of the article that faces inwardly toward the wearer and contacts the wearer is shown facing upward;

FIG. 4 is a section view of the absorbent article illustrated in FIGS. 1 and 2 taken along line 4-4 prior to being formed into a pull-on pant, wherein the interior portion of the article that faces inwardly toward the wearer and contacts the wearer is shown facing upward;

FIG. 5 is a section view of the absorbent article illustrated in FIGS. 1 and 2 taken along line 5-5 prior to being formed into a pull-on pant, wherein the interior portion of the article that faces inwardly toward the wearer and contacts the wearer is shown facing upward;

FIG. 6A is a simplified left side elevation view of an exemplary pull-on absorbent article showing the absorbent article worn about a lower torso region of a wearer;

FIG. 6B is a simplified right side elevation view of the pull-on absorbent article illustrated in FIG. 6A showing the absorbent article worn about the lower torso region of the wearer;

FIG. 6C is a front elevation view of the absorbent article illustrated in FIGS. 6A-B being worn about the lower torso region of the wearer;

FIG. 6D is a back elevation view of the absorbent article illustrated in FIGS. 6A-B being worn about the lower torso region of the wearer;

FIG. 7A is a schematic perspective view of the absorbent article illustrated in FIG. 1 configured as a pull-on absorbent article showing closed side interfaces constructed in accordance with one embodiment of the present invention;

FIG. 7B is a schematic perspective view of the absorbent article illustrated in FIG. 1 configured as a pull-on absorbent article showing closed side interfaces constructed in accordance with an alternative embodiment;

FIG. 7C is a schematic perspective view of the absorbent article illustrated in FIG. 1 configured as a pull-on absorbent article showing closed side interfaces constructed in accordance with an alternative embodiment;

FIG. 8 is a schematic view of a plurality of prepackaged pull-on absorbent articles constructed in accordance with the present invention;

FIG. 9 is a plan view of an exemplary fragment of a formed web material;

FIG. 10A is a plan view of an absorbent article similar to that illustrated in FIG. 1 but incorporating an elastic waist member;

FIG. 10B is a plan view of an absorbent article similar to FIG. 10A but with the elastic waist member constructed in accordance with an alternative embodiment;

FIG. 10C is a plan view of an absorbent article similar to FIG. 10A but with the elastic waist member constructed in accordance with another alternative embodiment;

FIG. 11 is a perspective view of an exemplary absorbent article prior to being formed into a pull-on pant shown in its relaxed, contracted state and with its interior portion facing upward;

FIG. 12 is a plan view of an exemplary absorbent assembly shown separately from the other portions of an exemplary absorbent article and with its interior portion facing the viewer;

FIG. 13 is a section view of the absorbent assembly illustrated in FIG. 12 taken along line 13-13;

FIG. 14 is a section view of the absorbent assembly illustrated in FIG. 12 taken along line 14-14;

FIG. 15 is a section view of an exemplary absorbent assembly showing details of an exemplary absorbent core having particles of superabsorbent material contained inside pockets;

FIG. 16 is a section view of an exemplary absorbent assembly having an additional bottom sheet taken at a section line corresponding to the section line 4-4 in FIG. 1;

FIG. 17 is a plan view of another exemplary disposable absorbent article in the form of an absorbent article shown in its flat, uncontracted state prior to being formed into a pull-on pant and with its interior portion facing the viewer;

FIG. 18 is a plan view of another exemplary disposable absorbent article in the form of an absorbent article shown in its flat, uncontracted state prior to being formed into a pull-on pant and with its interior portion facing the viewer;

FIG. 19 is a plan view of the absorbent article illustrated in FIG. 18 shown in its flat, uncontracted state prior to being formed into a pull-on pant with its exterior portion facing the viewer;

FIG. 20 is a section view of the absorbent article illustrated in FIGS. 18 and 19 taken at the section line 20-20, with its interior portion facing upward;

FIG. 21 is a plan view of another exemplary disposable absorbent article in the form of an absorbent article shown in its flat, uncontracted state prior to being formed into a pull-on pant and with its interior portion facing the viewer; and

FIG. 22 is a plan view of an exemplary disposable absorbent article similar to FIG. 18 but also further illustrating the absorbent assembly, wherein the backsheet strips and absorbent assembly are extensible and the absorbent article is illustrated in a simulated stretch configuration to illustrate lines of tension.

### DETAILED DESCRIPTION OF THE INVENTION

In this description, the following terms have the following meanings:

The term “absorbent article” refers to a device that absorbs and contains liquid, and more specifically, refers to a device that is placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or refastenable pant-type diapers, incontinence briefs and undergarments, diaper holders and liners, feminine hygiene garments such as panty liners, absorbent inserts, and the like.

The term “diaper” refers to an absorbent article that is generally worn by infants and incontinent persons about the lower torso so as to encircle the waist and the legs of the wearer and that is specifically adapted to receive and contain urinary and fecal waste.

The term “closed side interface” refers to a given side edge (or region adjacent the side edge), wherein a portion of the side edge (or region adjacent the side edge) in the front waist region is joined to a portion of the same side edge (or region adjacent the side edge) in the rear waist region on both sides of an absorbent article to define a closed, encircled leg openings and a closed waist opening. The side interface can be closed with a refastenable or permanent closure member.

The term “pant” (also referred to as “training pant”, “closed diaper”, and “pull-on diaper”) refers to disposable garments having a contiguous perimeter waist opening and contiguous perimeter leg openings designed for infant or adult wearers. A pant can be configured with a contiguous or closed waist opening and at least one contiguous, closed, leg opening prior to the article being applied to the wearer for use. A pant can be preformed by any suitable technique including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened, front waist fastened, rear waist fastened). Examples of suitable pants are disclosed in U.S. Pat. No. 5,246,433; U.S. Pat. No. 5,569,234; U.S. Pat. No. 6,120,487; U.S. Pat. No. 6,120,489; U.S. Pat. No. 4,940,464; U.S. Pat. No. 5,092,861; U.S. Pat. No. 5,897,545; U.S. Pat. No. 5,957,908; and U.S. patent Publication No. 2003/0233082 A1.

The term “closure member” refers to an element that maintains the article waist and leg openings in a closed, contiguous, configuration until the closure member is released. Suitable closure members include a seam, an adhesive, a cohesive, a heat bond, a pressure bond or weld, a tab-and-slot configuration, a hook-and-loop configuration, and the like.

The term “refastenable closure member” refers to a closure member that can be opened and subsequently re-closed, reliably, without destroying the closure member or surrounding diaper components. Examples of refastenable closure members include peelable adhesives, cohesives, and the like, and mechanical fasteners such as tabs-and-slots, hooks-and-loops, and the like.

The term “permanent closure member” refers to a closure member that cannot be opened without causing the closure member to fail (i.e., the closure member cannot again be reliably closed). At times, when attempting to open a permanent closure member, surrounding absorbent article component(s) may be damaged or torn. Examples of permanent closure members include adhesives, heat bonds, pressure welds, cohesives, and the like, and further include seams.

The term “seam” refers to an elongated line of junction that attaches two regions of the backsheet strips of a diaper. Seams can be created via thermal bonding, pressure bonding, ultrasonic bonding, adhesive bonding, welds, and stitching. A seam is typically configured as a permanent closure member.

The term “pre-closed” refers to an absorbent article that can be closed by the end user and formed into a pant-like garment prior to applying the garment to the wearer. The term “pre-closed” also encompasses an absorbent article that has been formed into a pant-like garment in the packaging 161 such that the end user receives the article as a pant-like garment that can be directly applied to the wearer.

The term “cohesive” refers to the property of a material that sticks to itself but does not to any significant degree stick to other materials.

The term “disposable” refers to the nature of absorbent articles that generally are not intended to be laundered or otherwise restored or reused as an absorbent article, i.e., they are intended to be discarded after a single use and, preferably, to be recycled, composted or otherwise disposed of in an environmentally compatible manner.

The term “extensible” refers to any material which, upon application of a biasing force of less than 500 grams/inch is elongatable, at least about 20 percent without experiencing catastrophic failure.

The term “longitudinal” refers to a direction running from a waist edge to an opposing waist edge of the article and generally parallel to the maximum linear dimension of the article. Directions within ±45° of the longitudinal direction are considered to be “longitudinal”.

The term “lateral” refers to a direction running from a side edge to an opposing side edge of the article and generally at a right angle to the longitudinal direction. Directions within ±45° of the lateral direction are considered to be “lateral”.

The term “disposed” refers to an element or region being attached and/or positioned in a particular place or position in a unitary structure with other elements.

The term “attached” refers to elements being connected or united by fastening, adhering, bonding, etc. by any method suitable for the elements being attached together and their constituent materials. Many suitable methods for attaching elements together are well-known, including adhesive bonding, pressure bonding, thermal bonding, mechanical fastening, etc. Such attachment methods can be used to attach elements together over a particular area either continuously or intermittently.

The terms “water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water to pass through its thickness in the absence of a forcing pressure. Conversely, the term “water-impermeable” refers to a layer or a layered structure through the thickness of which liquid water cannot pass in the absence of a forcing pressure. A layer or a layered structure that is water-impermeable according to this definition can be permeable to water vapor, i.e., can be “water vapor-permeable”. Such a water vapor-permeable layer or layered structure is commonly known in the art as “breathable”. As is well known in the art, a common method for measuring the permeability to water of the materials typically used in absorbent articles is a hydrostatic pressure test, also called a hydrostatic head test or simply a “hydrohead” test. Suitable well known compendial methods for hydrohead testing are approved by INDA (formerly the International Nonwovens and Disposables Association, now The Association of the Nonwoven Fabrics Industry) and EDANA (European Disposables And Nonwovens Association).

The terms “proximal” and “distal” refer respectively to the location of an element relatively near to or far from the center of a structure, e.g., the proximal edge of a longitudinally extending element is located nearer to the longitudinal axis than the distal edge of the same element is located relative to the same longitudinal axis.

The terms “interior” and “exterior” refer respectively to the location of an element that is intended to be placed against or toward the body of a wearer when an absorbent article is worn and the location of an element that is intended to be placed against or toward any clothing that is worn over the absorbent article. Synonyms for “interior” and “exterior” include, respectively, “inner” and “outer”, as well as “inside” and “outside”. Also, when the absorbent article is oriented such that its interior faces upward, e.g., when it is laid out in preparation for setting the wearer on top of it, synonyms include “upper” and “lower” and “top” and “bottom”, respectively.

Description of Exemplary Diaper Embodiments

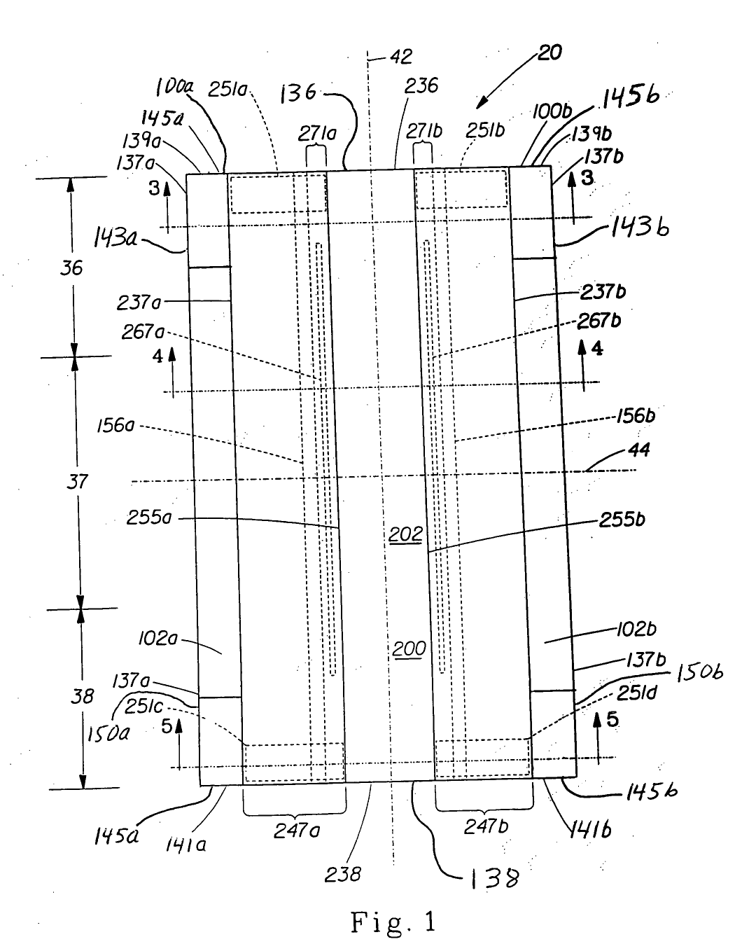

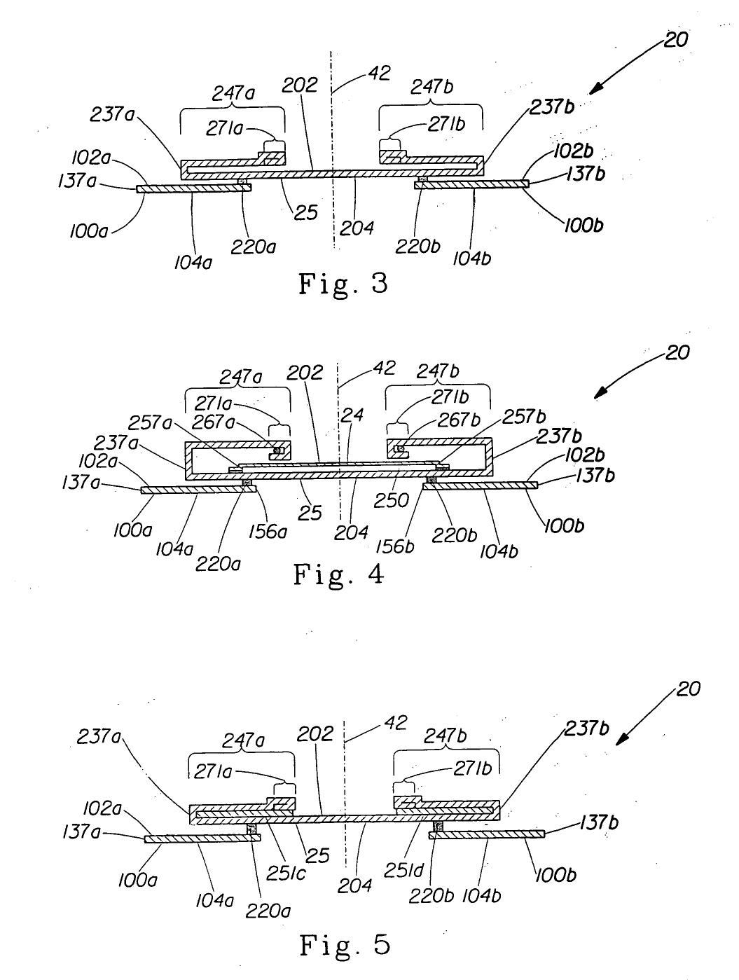

As shown in FIGS. 1-5, one end portion of an absorbent article, illustrated as an exemplary pant-like garment, also referred to as a pant or pull-on diaper 20, defines a first longitudinal end portion configured as a front waist region 36, a longitudinally opposing end portion configured as a back waist region 38, and an intermediate portion configured as a crotch region 37.

The basic structure of the pull-diaper 20 includes an absorbent assembly 200, which has a front edge 236, a back edge 238, a left side edge 237a, a right side edge 237b, an interior surface 202, and an exterior surface 204. A longitudinal axis 42 extends through the midpoints of the front edge 236 and the back edge 238 and a lateral axis 44 extends through the midpoints of the left side edge 237a and the right side edge 237b. The absorbent assembly 200 has a lower covering sheet 25 and laterally opposing side flaps 247a and 247b. The covering sheet 25 and side flaps 247 are described in more detail below.

The basic structure of the pull-on diaper 20 also includes two laterally opposing longitudinally extending backsheet strips including a left backsheet strip 100a and the right backsheet strip 100b. Each of the backsheet strips 100 has a respective left front waist edge 139a and a right front waist edge 139b, a left back waist edge 141a and a right back waist edge 141b, a left strip proximal edge 156a and a right strip proximal edge 156b, a left interior surface 102a and a right interior surface 102b, and a left exterior surface 104a and a right exterior surface 104b. Together, the two backsheet strips 100 define a left side edge 137a and a right side edge 137b of the pull-on diaper 20. The left and right front waist edges 139 of the backsheet strips 100, in combination with the front edge 236 of the absorbent assembly 200, define a front waist edge 136 of the pull-on diaper 20. Likewise, the left and right back waist edges 141 of the backsheet strips 100, in combination with the back edge 238 of the absorbent assembly 200, define a back waist edge 138 of the pull-on diaper 20.

The backsheet strips 100 can be formed of a nonwoven material, for example a synthetic nonwoven such as spunbonded or carded polyethylene, polypropylene, polyester, or rayon. Alternatively or in addition, each backsheet strip 100a and 100b can include a water-impermeable layer that is formed of a suitable material, for example a film of polyethylene or another polyolefin, a microporous breathable film, a hydrophobic nonwoven, or a film formed of coextruded polyolefin layers. For example, a suitable coextruded film is available from Clopay Plastic Products Co. of Mason, Ohio, U.S.A. under the designation of M18-327. A multi-layer backsheet strip, such as a laminate of a film and a nonwoven, can also be suitable and can be oriented with the nonwoven disposed exteriorly to provide the feel and appearance of a cloth-like outermost layer, or alternatively with the nonwoven disposed interiorly to separate the film from the skin of the wearer, or with nonwovens disposed both exteriorly and interiorly.

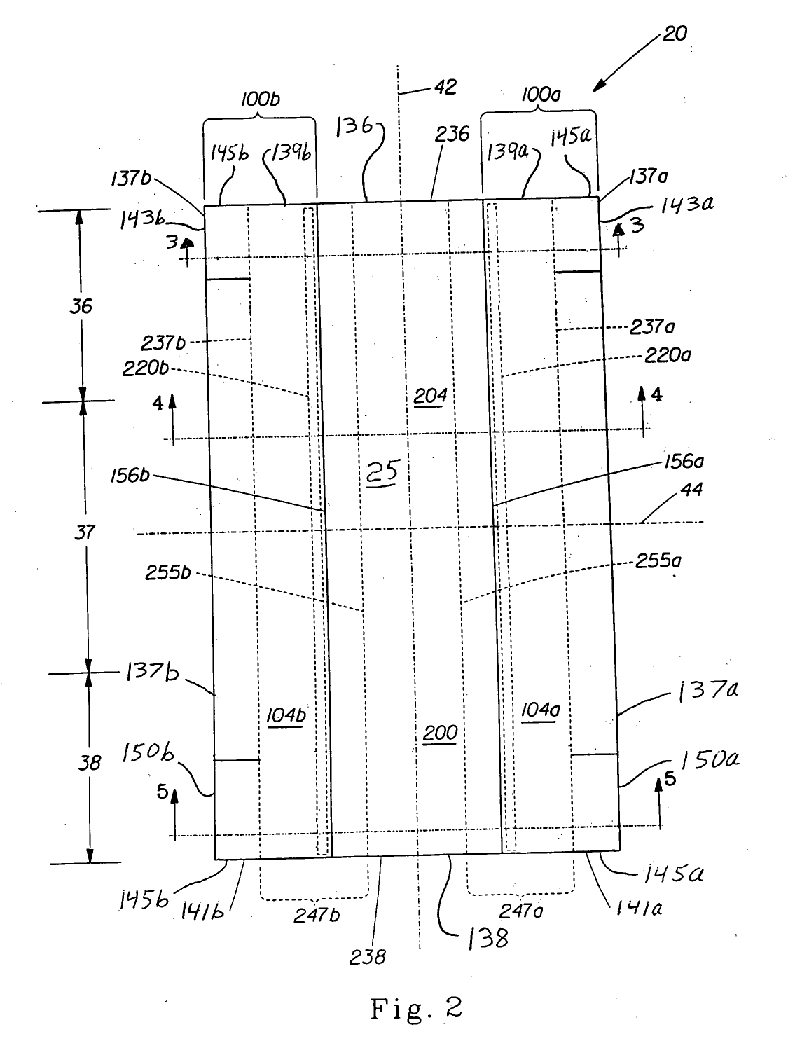

As shown in FIGS. 2-5, the backsheet strips 100a and 100b and the lower covering sheet 25 of the absorbent assembly 200 are attached together in laterally opposing longitudinally extending attachment zones such as the exemplary attachment zones 220a and 220b.

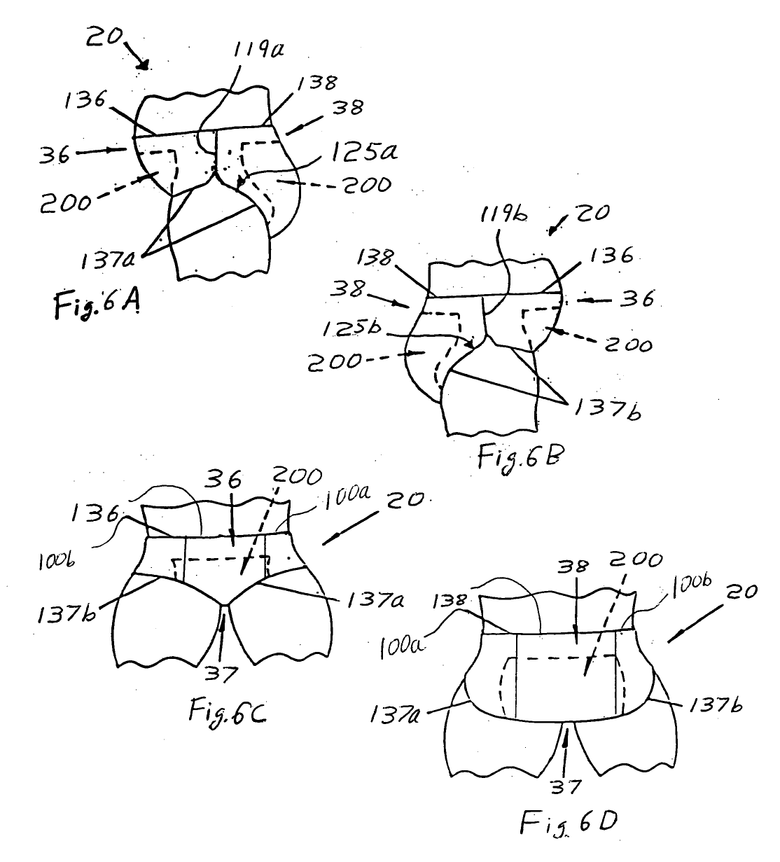

As shown in FIGS. 6A-6D, when the pull-on diaper 20 is worn on the lower torso of a wearer, the front waist edge 136 and the back waist edge 138 combine to encircle the waist of the wearer while, at the same time, the backsheet strip side edges 137a and 137b encircle the legs of the wearer, and thus define left and right leg openings 125a and 125b, respectively. The crotch region 37 is generally positioned between the legs of the wearer, such that the absorbent assembly 200 extends from the front waist region 36 through the crotch region 37 to the back waist region 38.

As illustrated in FIGS. 1 and 6A-B, a garment can be pre-formed by the manufacturer to create a pull-on diaper or pant 20. Specifically, the pull-on diaper 20 includes left and right closed side interfaces 119a and 119b, each disposed at left and right side edge regions 145a and 145b, respectively, which define regions adjacent the respective side edge 137a and 137b and are disposed in the waist regions 36 and 38. The closed side interfaces 119a and 119b can also include their respective side edges 137a and 137b. The side edge regions 145a and 145b can extend as longitudinally inward from front and back waist edges 136 and 138 as desired. The closed left side interface 119a is defined by an attachment between 1) the left side edge region 145a at a front left attachment zone 143a disposed in the front waist region 36, and 2) the left side edge region 145a at a back left attachment zone 150a disposed in the back waist region 38. Similarly, the closed right side interface 119b is defined by an attachment between 1) the right side edge region 145b at a front right attachment zone 143b disposed in the front waist region 36, and 2) the right side edge region 145b at a back attachment zone 150b disposed in the back waist region 38. The attachment zones 143a-b may or may not extend to the corresponding waist edges 136 and 138, and may or may not extend to the corresponding side edges 137a and 137b. Furthermore, one skilled in the art will appreciate that the attachment zones 143a-b could be closed using any permanent or refastenable closure member. The attachment zones 143a-b at the side edge regions 145a-b can be attached to form closed side interfaces 119a-b by buttressing and subsequently attaching the side edge 137a in the front and back waist regions 36 and 38, and side edge 137b in the front and back waist regions 36 and 38, respectively, either using a permanent or refastenable closure member, as illustrated in FIGS. 6A-B.

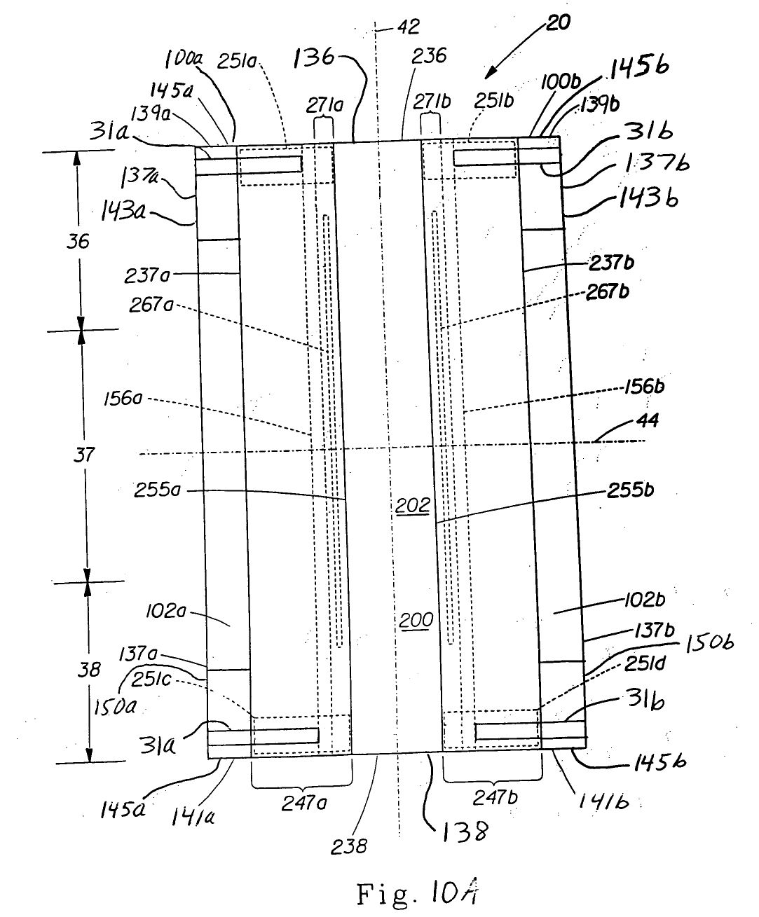

Because the diaper 20 is configured as a pull-on diaper, both side interfaces 119a and 119b are pre-closed, meaning that the side interfaces 119a-b are closed prior to removal of the pull-on diaper 20 from its package 161, as illustrated in FIG. 8, and therefore prior to being donned on the wearer. The closed side interfaces 119a-b, in part, define the contiguous, closed, left and right leg openings 125a and 125b, respectively, and a contiguous, closed, waist opening 144, adapted to fit and gasket the wearer's legs and waist, respectively, as the pull-on diaper 20 is pulled up to the wearer's lower torso region. The side interfaces 119a-b can be formed into a closed configuration in accordance with any known techniques or methods known in the art. For instance, the interfaces 119a and 119b can be formed with a permanent seam, which can include a bond formed by heat sealing such as ultrasonic bonding, high pressure bonding, RF (radio frequency) bonding, hot air bonding, heated point bonding, and the like as appreciated by one having ordinary skill in the art. Various suitable pant configurations are disclosed in U.S. Pat. No. 5,246,433 (issued on Sep. 21, 1993 to Margeret H. Hasse, et al); U.S. Pat. No. 5,569,234 (issued on Oct. 29, 1996 to Kenneth B. Buell, et al); U.S. Pat. No. 6,120,487 (issued on Sep. 19, 2000 to Gregory Ashton); U.S. Pat. No. 6,120,489 (issued on Sep. 19, 2000 to Larry Johnson, et al); U.S. Pat. No. 4,940,464 (issued on Jul. 10, 1990 to Paul T. Van Gompel); U.S. Pat. No. 5,092,861 (issued on Mar. 3, 1992 to Hironori Nomura et al); U.S. Pat. No. 5,897,545 (issued on Apr. 27, 1999 to Mark James Kline, et al); U.S. Pat. No. 5,957,908 (issued on Sep. 28, 1999 to Mark James Kline, et al); and U.S. patent Publication No. 2003/0233082 A1 (published on Dec. 18, 2003 to Mark J. Kline, et al).

Alternatively, the closed side interfaces 119a-b can be formed as disclosed in U.S. Pat. No. 5,779,831 (issued on Jul. 14, 1998 to Christoph Schmitz); U.S. Pat. No. 5,772,825 (issued on Jun. 30, 1998 to Christoph Schmitz); U.S. Pat. No. 5,607,537 (issued on Mar. 4, 1997 to Larry Johnson, et al); U.S. Pat. No. 5,622,589 (issued on Apr. 22, 2997 to Larry Johnson, et al); U.S. Pat. No. 5,662,638 (issued on Sep. 2, 1997 to Larry Johnson, et al); U.S. Pat. No. 6,042,673 (issued on Mar. 28, 2000 to Larry Johnson, et al); and U.S. Pat. No. 6,726,792 (issued on Apr. 27, 2004 to Larry Johnson, et al). The aforementioned patents disclose various processing methods to provide absorbent pull-on diapers. One of the processes utilizes a final knife followed by a reciprocating tucker blade that pushes the pad from a horizontal orientation to a vertical orientation and a vacuum conveyor belt that holds the pad through a high pressure side seaming unit. The side seaming unit is followed by a slitter that trims the pant edges to provide a finished seam edge. An alternative method disclosed in the aforementioned patents involves cutting the pad in the final knife and bi-folding the pad collecting the pads in a “waterwheel” stacker (a rotary slotted wheel). The bonding is accomplished while the pad is held in place on the rotating wheel.

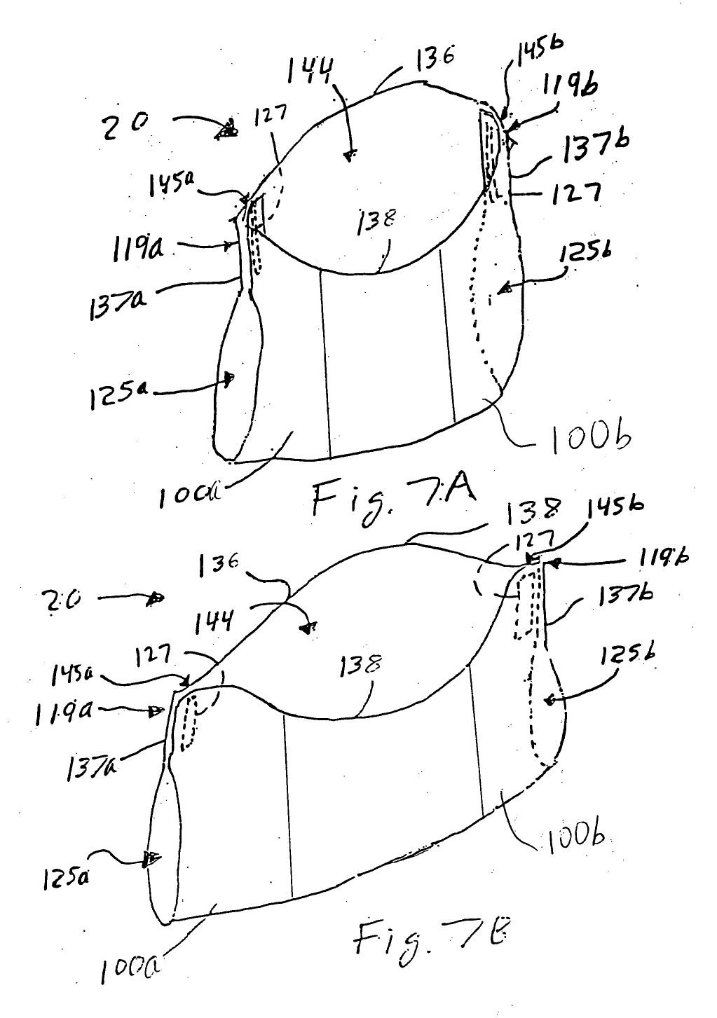

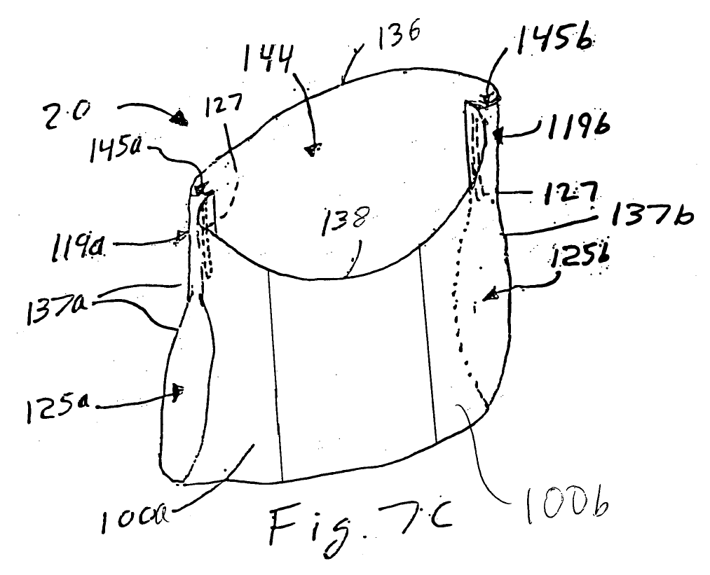

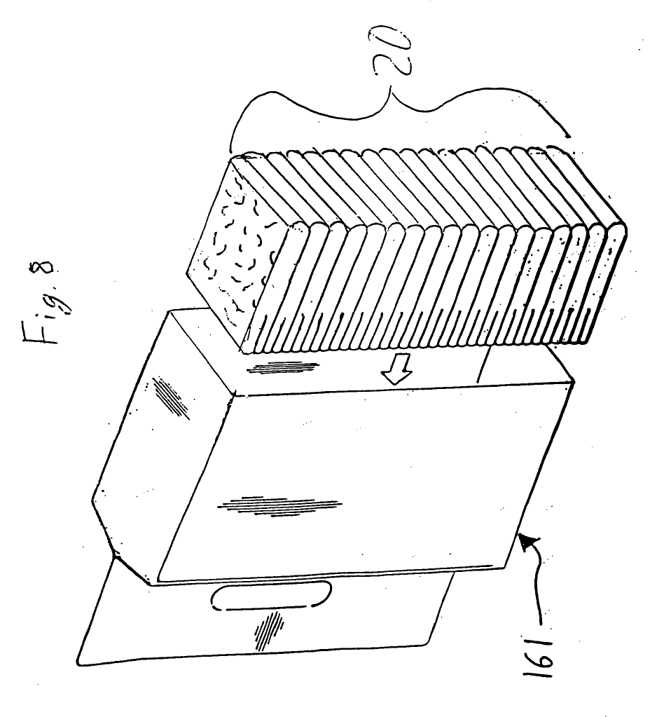

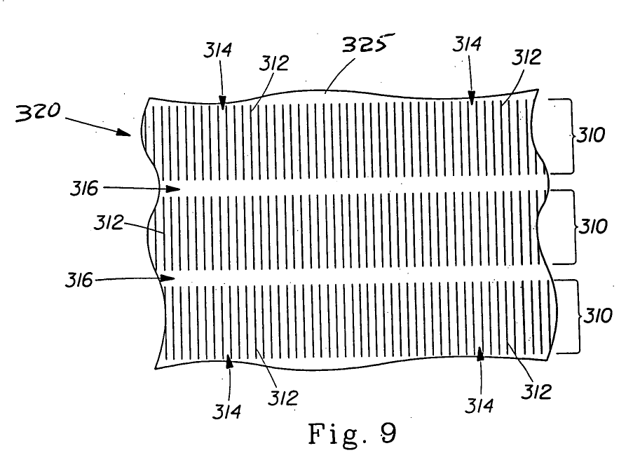

Alternatively, referring to FIGS. 1 and 7A, a left side edge region 145a (defined as a region adjacent the left side edge 137a and including the left side edge 137a) at the front left attachment zone 143a (i.e., in the front waist region 36) is overlapped with the left side edge region 145a at the back attachment zone 150a (i.e., in the back waist region 38) in an interior surface-to-exterior surface (or vice versa) configuration. Likewise, a right side edge region 145b (defined as a region adjacent the right side edge 137b and including the right side edge 137b) at the front right attachment zone 143b (i.e., in the front waist region 36) is overlapped with the right side edge region 145b at the back attachment zone 150b (i.e., in the back waist region 38) in an interior surface-to-exterior surface (or vice versa) configuration. Accordingly, the left and right side interfaces 119a and 119b can be closed by attaching the overlapping attachment zones 143 and 150 via any suitable permanent or refastenable closure member 127, such as a seam of the type described above, or an adhesive, a cohesive, a tab-and-slot configuration, or via hook-and-loop attachments. It should be appreciated that joining the side edge regions 145a and 145b causes the side edges 137a and 137b to correspondingly be joined indirectly via the side edge regions 145a and 145b.

Alternatively, referring to FIGS. 1 and 7B, the closed side interfaces 119a and 119b are formed by bi-folding the backsheet strips 100 such that the left and right side edge regions 145a-b, adjacent the front waist edge 136, overlap the left and right side edge regions 145a-b, respectively, adjacent the back waist edge 138 in an interior-to-interior surface configuration. In this configuration, the front waist edge 136 can be substantially aligned with the back waist edge 138 and the side edge 137a in the front and back waist regions can also be substantially aligned as can be the front and back waist regions of side edge 137b. The folded backsheet strips 100 are then attached at the side edge regions 145a-b at the attachment zones 143 and 150, respectively (FIG. 1), using any suitable permanent or refastenable closure member 127, thereby forming a pull-on diaper defining contiguous left and right leg openings 125a and 125b, respectively, and a contiguous, closed, waist opening 144.

Alternatively still, referring to FIGS. 1 and 7C, the closed side interfaces 119a and 119b can be formed by bi-folding backsheet strips 100 such that the left and right side edge regions 145a-b, adjacent the front waist edge 136, overlap the left and right side edge regions 145a-b, respectively, adjacent the back waist edge 138 in an exterior-to-exterior surface configuration. In this configuration, the front end edge 136 can be substantially aligned with the back end edge 138. The folded backsheet strips 100 then attached at the side edge regions 145a-b at the attachment zones 143 and 150, respectively (FIG. 1), using any suitable permanent or refastenable closure member 127, thereby forming a pull-on diaper defining contiguous left and right leg openings 125a and 125b, respectively, and a contiguous, closed, waist opening 144.

Furthermore, one having ordinary skill in the art will appreciate that the side interfaces 119a-b can be closed via a refastenable closure member that can be nondestructively opened and refastened. Examples of refastenable closure members include hook-and-loop fasteners, snaps, tab-slot fasteners, cohesives, and the like.

Examples of closure members are described in U.S. Pat. No. 6,432,098 (issued Aug. 13, 2002 to Kline et al); U.S. Pat. No. 6,880,211 (issued Apr. 19, 2005 to Jackson et al); and U.S. patent Publication No. 2003/0233082 (published Dec. 18, 2003 to Kline et al).

The present invention therefore recognizes that a plurality of pull-on diapers 20 can be pre-formed having the closed side interfaces 119a and 119b packaged, and subsequently delivered to a user to prevent the need for the user (which could be the wearer) to close the side edges 137a and 137b prior to securing the pull-on diaper 20 on the wearer. Accordingly, referring to FIG. 8, the present invention includes the method of providing a plurality of pull-on diapers 20 of the type described above, placing the diapers 20 into a closed package or other containment apparatus 161 that retains the diapers 20. Accordingly, when the end user opens the packaging 161, the pull-on diaper 20 can be donned on the wearer more easily than conventional taped diapers.

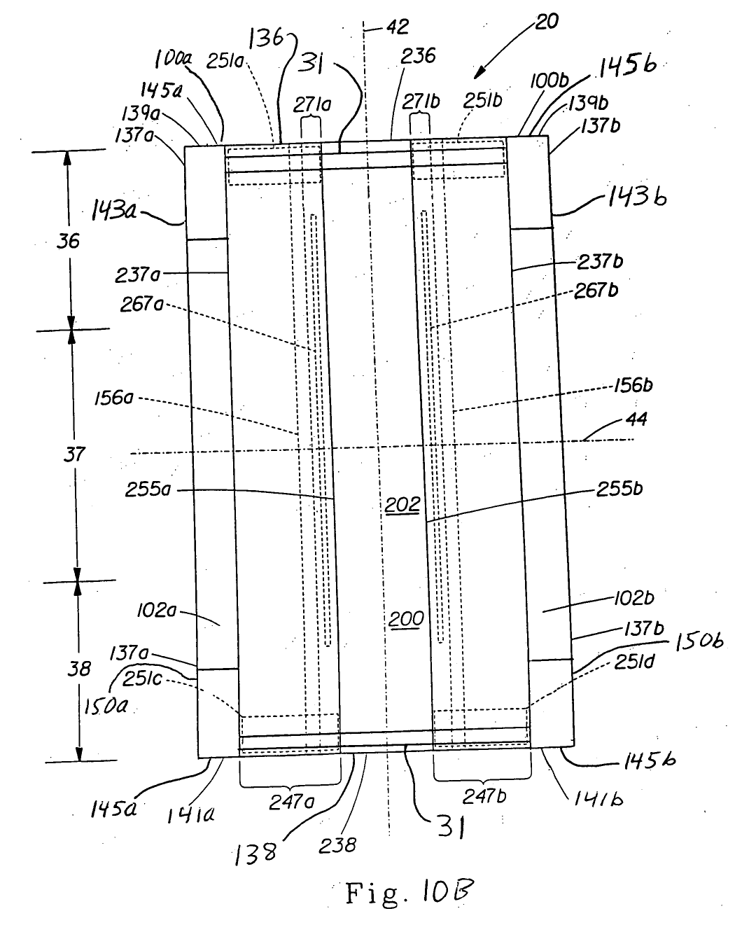

A portion or the whole of each of the backsheet strips 100 can be formed of an elastically extensible material or materials. Alternatively, or in addition, a portion or the whole of each of the backsheet strips 100 can be made extensible to a degree greater than the inherent extensibility of the material or materials from which the backsheet strip is made. Similarly, a portion or the whole of the absorbent assembly 200 can be formed of an elastically extensible material or materials. Alternatively or in addition, a portion or the whole of the absorbent assembly 200 can be made extensible to a degree greater than the inherent extensibility of the material or materials from which the absorbent assembly 200 is made. Advantageously, the pull-on diaper 20 can exhibit an elastic-like behavior in the direction of elongation without the use of added elastic materials. The elastic-like behavior can be modified and/or provided as desired in a web material 325 (FIG. 9) as described below. The additional extensibility may be desirable in order to allow the pull-on diaper 20 to conform to the body of a wearer during movement by the wearer. The additional extensibility may also be desirable, for example, to allow the user of a pull-on diaper 20 including backsheet strips 100 having a particular size before extension to extend the front and/or back waist regions 36 and 38 to enable the pull-on diaper to be pulled over the hips of the wearer and then to contract to encircle the waist of an individual wearer whose waist circumference is typically smaller than the circumference as measured at the hips of the wearer. Such a lateral extension of the waist region(s) can give the diaper 20 a generally hourglass shape, so long as the crotch region 37 is extended to a relatively lesser degree than the waist region(s), and can impart a tailored appearance to the pull-on diaper 20 when it is worn. In addition, the additional extensibility may be desirable in order to minimize the cost of the pull-on diaper 20. Specifically, a lesser amount of material is needed in order to make a diaper capable of being properly fit onto a given size of a wearer when the material is made extensible as described.

For the purpose of fitting to the waist of the wearer, in some embodiments additional lateral extensibility in the absorbent assembly 200 can be provided between the laterally opposing attachment zones 220a and 220b where the absorbent assembly 200 and the backsheet strips 100a and 100b are attached together, rather than in the entire absorbent assembly 200.

Additional extensibility in the lateral direction is relatively more useful than additional extensibility in the longitudinal direction. The abdomen of the wearer is likely to expand when the wearer changes posture from standing to sitting and the corresponding abdominal expansion increases the circumference that is encircled by the waist edges 236 and 238, rendering lateral extension of the waist region or regions particularly advantageous.

Additional extensibility in the backsheet strips 100 and/or the absorbent assembly 200 can be provided in a variety of ways. For example, a material or materials from which the backsheet strips and/or the absorbent assembly is/are made can be pleated by any of many known methods. Alternatively, all or a portion of the backsheet strips 100 and/or the absorbent assembly 200 can be made of a formed web material or a formed laminate of web materials like those described in U.S. Pat. No. 5,518,801 (issued May 21, 1996 to Chappell et al), U.S. Pat. No. 5,691,035 (issued Nov. 25, 1997 to Chappell et al), U.S. Pat. No. 5,723,087 (issued Mar. 3, 1998 to Chappell et al), U.S. Pat. No. 5,891,544 (issued Apr. 6, 1999 to Chappell et al), and U.S. Pat. No. 5,968,029 (issued Jan. 19, 1999 to Chappell et al). An exemplary fragment 320 of such a formed web material 325 is shown in FIG. 9. This formed web material 325 includes distinct laterally extending regions 310 in which the original material has been altered by embossing or another method of deformation to create a pattern of generally longitudinally oriented alternating ridges 312 and valleys 314. The formed web material 325 also includes laterally extending unaltered regions 316 located between the laterally extending altered regions 310.

Such a formed web material 325 can be laterally extended beyond its original dimension with the application of relatively less force than that required to extend the same material to the same extent when undeformed. In particular, the application of opposing divergent forces directed generally perpendicular to the ridges 312 and valleys 314 extends such a formed web material along an axis between the opposing forces and generates a resistive contractive force, primarily in the unaltered regions 316. This resistive force is relatively smaller than the resistive force that is generated by the same material in its unaltered form when extended to the same extent, at least up to an extension at which the ridges and valleys in the altered regions flatten and begin to contribute to the resistive force. Thus, such formed web materials exhibit an extensible behavior resembling that of traditional elastic materials in the range of extensibility that is useful in absorbent articles, but can be made of relatively less expensive materials that are not inherently elastic and, thus, their use may provide an advantage in terms of the cost of manufacturing the absorbent articles.

The range of extensibility of a web material or a laminate that is formed as described in the Chappell et al. '801 patent can be controlled by the degree of deformation of the altered regions and can be varied from near zero to a maximum that is dependent upon the original material. For example, the materials used in the backsheet strips 100 of the exemplary pull-on diaper 20 can typically be formed to provide any range of extensibility from a minimum of 20% to a maximum of more than 100 percent of the original dimension. In some embodiments of the present invention, a portion of the backsheet strips 100 can have a level of extensibility within a range whose lower end is defined by and between 20%, 25%, and 30%, and whose upper end is defined by and between 40%, 60%, and 80%. The requisite levels of extensibility are achieved by application of an opposing divergent force in the direction of extensibility of preferably less than 1,000 grams/inch, more preferably less than 700 grams/inch. However, it should be easily appreciated that any particular value for the maximum extensibility in the range from approximately twenty percent to approximately 100 percent can be selected to suit a particular choice of the original size of the diaper 20 and the range of sizes of the intended wearers. In particular, a diaper having a specific unextended waist opening circumference can be suitable for use on wearers having waist circumferences ranging from equal to this unextended waist opening circumference up to the maximum extensibility.

When the web 325 is subjected to an applied elongation, the web material exhibits an elastic-like behavior as it extends in the direction of applied elongation and returns to its substantially untensioned condition once the applied elongation is removed, unless the web material is extended beyond the point of yielding. The web extensibility is adjustable by varying the percentage of the web surface which is comprised of the ridges 312 and valleys 314. This can be achieved, for instance, by modifying the widths of the ridges 312 and valleys 314, and the spacing between adjacent ridges 312 and valleys 314. A higher percentage of area coverage of the web material 325 by the ridges 312 and valleys will increase the overall extensibility of the web 325. The web 325 is able to undergo multiple cycles of applied elongation up to the yield point without losing its ability to substantially recover. Accordingly, the web 325 is able to return to its substantially untensioned condition once the applied elongation is removed (e.g., as the diaper 20 is pulled over the wearer's waist region during use).

In addition, different portions of the backsheet strips and/or the absorbent assembly can be formed to have different ranges of extensibility and/or to be extensible to a greater or lesser degree when subjected to a given level of opposing tensile forces, i.e., to be relatively more easily or less easily extensible. Such differential extensibility may be desirable so that, for example, one or both of the waist regions can be laterally extended relatively farther or relatively more easily than the crotch region.

Any of a variety of extensible materials can be formed as described in the Chappell et al. '801 patent. For example, a film, a nonwoven, or a laminate of either or both of these materials can be formed to provide the desired extensibility. It is also possible to modify such a material in more than one way while forming it to provide extensibility. For instance, a film that is originally formed to resist the permeation of vapor through its thickness and to contain fine particles of a granular filler material such as calcium carbonate can be treated as described in the Chappell et al. '801 patent to simultaneously provide extensibility and create small holes that allow water vapor to pass through its thickness. Thus, the film can simultaneously be rendered extensible and breathable.

Alternatively, a portion of the lower covering sheet 25 or backsheet strips 100a and 100b can be ring-rolled and thus rendered highly extensible as described in U.S. Pat. No. 5,366,782 (issued Nov. 22, 1994 to Curro, et al). Specifically, a ring-rolling apparatus includes opposing rolls having intermeshing teeth that incrementally stretch and thereby plastically deform the material forming lower covering sheet 25 (or a portion thereof) thereby rendering the lower covering sheet 25 extensible in the ring-rolled regions. In one embodiment, the lower covering sheet 25 can be ring-rolled in a portion of at least one of the front or back waist regions while other regions may comprise a structured elastic-like formed web material. The backsheet strips 100 and/or absorbent assembly 200 may be ring-rolled across their entire width in one or both of the waist regions or alternatively may be ring-rolled over only a portion of their width.

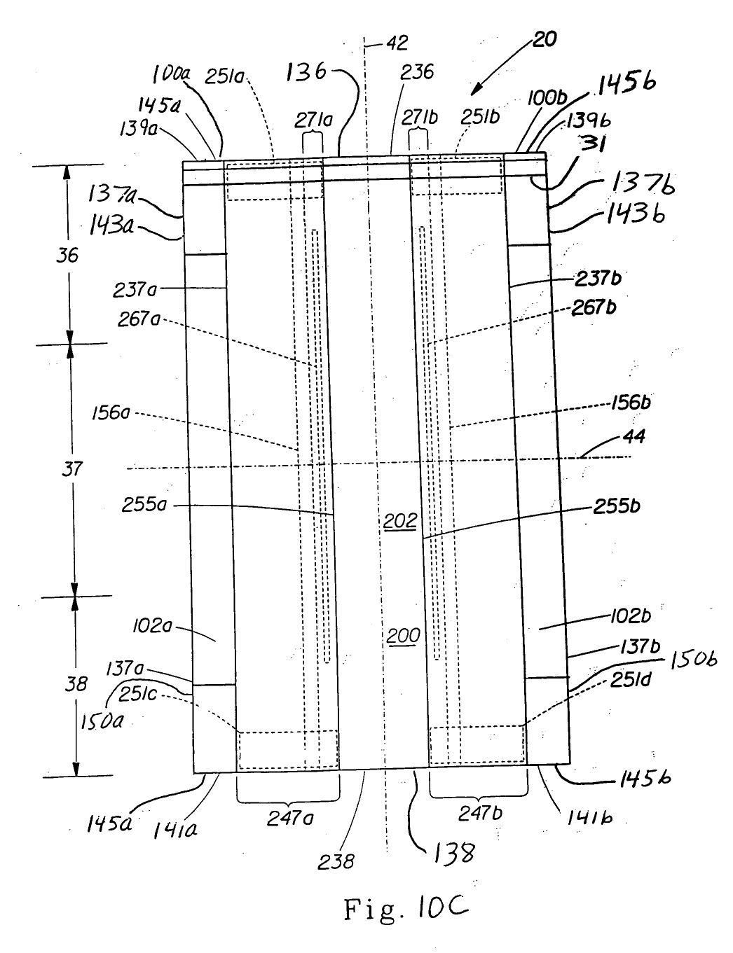

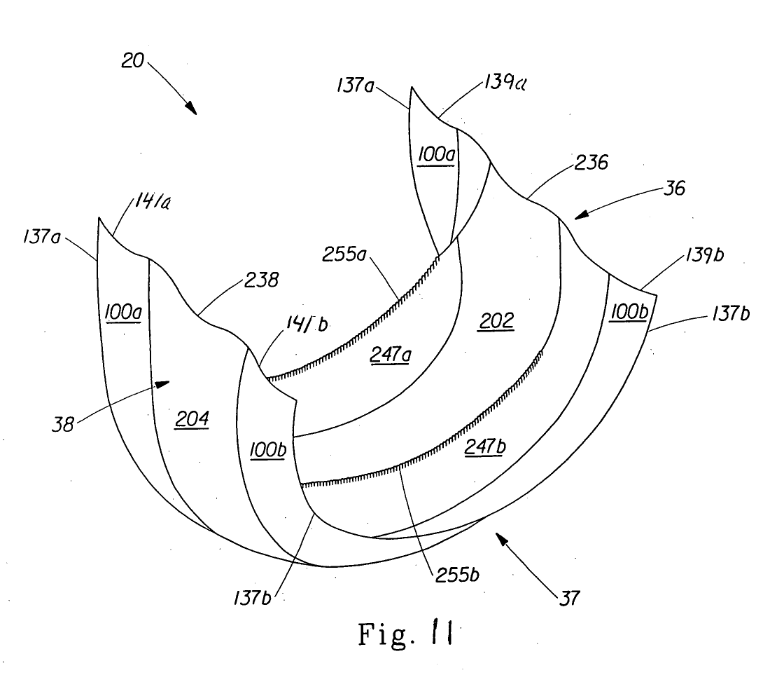

Referring now to FIGS. 10A-C, certain aspects of the present invention recognize that the pull-on diaper 20 can include at least one fit enhancement member, illustrated as an elongated elastic waist member 31 configured to provide a contractive force to the absorbent assembly 200 and/or the backsheet strips 100 at the front waist region 36 and/or back waist region 38 adjacent the corresponding waist edges 136 and 138 (and hence at the wearer's waist region). The elastic waist member 31 comprises an elongated strip of material whose elongation has at least a lateral component (and optionally a longitudinal component) that can be formed from an extensible material or formed from a non-extensible material that is rendered extensible using techniques described in more detail below, such that the elastic waist member 31 can be laterally extensible. The elastic waist member 31 can be either pre-tensioned prior to attachment to the diaper 20 or non pre-tensioned. If pre-tensioned, the waist member 31 will apply a contractive force to the diaper that tends to reduce the circumference of the waist opening 144. If non pre-tensioned, the waist member 31 will not apply a contractive force until the diaper 20 is pulled over a structure (e.g., the wearer's hip region) sufficient to extend the waist member 31.

Referring now to FIG. 10A, two pair of elastic waist members 31a and 31b are provided. The first pair of elastic waist members 31a extends from the left side edge 137a to the left strip proximal edge 156a adjacent the left front and back waist edges 139a and 141a, respectively. The second pair of elastic waist members 31b extends from the right side edge 137b to the right strip proximal edge 156b, respectively adjacent the right front and back waist edges 139b and 141b, respectively. The elastic members 31 can be attached to the barrier strips 100 in any suitable manner known to one having ordinary skill in the art. It should thus be appreciated that the elastic members 31 overlap the backsheet strips 100. This configuration is particularly advantageous when the backsheet strips 100 are non-extensible and the absorbent assembly 200 is extensible, though it should be appreciated that the elastic members 31 can be configured as illustrated in FIG. 10A when the absorbent assembly 200 is non-extensible, and furthermore when the absorbent assembly 200 and the backsheet strips 100 are both extensible. For configurations wherein the backsheet strips 100 are non-extensible the elastic member 31 could be applied to the backsheet strips 100 in a pre-tensioned condition. For configurations wherein the backsheet strips 100 are extensible, the elastic members 31 could be applied in a non pre-tensioned condition.

Referring now to FIG. 10B, an elastic member 31 extends between the side edges 237a and 237b of the absorbent assembly 200. It should thus be appreciated that the elastic member 31 overlaps the absorbent assembly 200. This configuration is particularly advantageous when the absorbent assembly 200 is non-extensible and the backsheet strips 100 are extensible, though it should be appreciated that the elastic member 31 can be configured as illustrated in FIG. 10B when the backsheet strips 100 are non-extensible, and furthermore when the absorbent assembly 200 and the elastic member 31 are both extensible. For configurations wherein the absorbent assembly is non-extensible the elastic member would be applied to the absorbent assembly in a pre-tensioned condition. For configurations wherein the absorbent assembly is extensible, the elastic member could be applied in a non pre-tensioned condition.

If the absorbent assembly 200 is non-extensible or plastically extensible, the overlying elastic waist member 31 is applied in a pre-tensioned condition and thereby will cause the diaper 20 at the region of the backsheet strips 100 to exert a contractive force on the wearer's waist while the diaper 20 is worn, thus improving the fit of the diaper 20 on the wearer. If, on the other hand, if the absorbent assembly 200 is extensible, the elastic member 31 is applied in a non pre-tensioned state such that when the waist opening 144 is extended to allow the diaper 20 to pass over the wearer's hip region during application, the elastic waist member 31 will exert a contractive force as the waist is released and contracts to fit the waist of the wearer thereby improving the fit of the diaper 20 on the wearer.

Referring now to FIG. 10C, the elastic member 31 extends from the left side edge 137a to the right side edge 137b, and can be attached to the backsheet strips 100 and/or the absorbent assembly 200 in any known manner. It should thus be appreciated that the elastic member 31 overlaps both the backsheet strips 100 and the absorbent assembly 200. For configurations wherein the backsheet strips 100 and the absorbent assembly 200 are non-extensible, it should be appreciated that the elastic member 31 can be applied in a pre-tensioned condition. For configurations wherein either the backsheet strips 100 or the absorbent assembly 200 is extensible the elastic member can be applied in a non pre-tensioned condition.

If the backsheet strips 100 and the absorbent assembly 200 are non-extensible or the pre-tensioned elastic waist member 31 will cause the diaper 20 at the region of the backsheet strips 100 and absorbent assembly 200 to exert a contractive force on the wearer's waist while the diaper 20 is worn, thus improving the fit of the diaper 20 on the wearer. If, on the other hand, the backsheet strips 100 and/or the absorbent assembly 200 are extensible, the elastic member will exert a contractive force once the waist has been extended sufficiently to allow the diaper 20 to pass over the wearer's hip region during application, the elastic waist member 31 will apply a contractive force thereby improving the fit of the diaper 20 on the wearer.

Once the diaper 20 has been positioned on the lower torso region of the wearer, the web 325 and/or the elastic member 31 enables the diaper 20 to apply a contractive force at the front and back waist regions 36 and 38, respectively, to the wearer's body at a level greater than 100 grams, alternatively greater than 200 grams, and alternatively still greater than 300 grams. It may also be desired that the diaper 20 applies a contractive force at the waist regions 36 and 38 that is less than 2,000 grams, alternatively less than 1,500 grams and alternatively still less than 1,000 grams. As described in the Chappell et al. '801 patent, the resistive force exerted by the web 325 (i.e., the contractive force) in response to an applied elongation can be modified. Specifically, the web and/or elastic member 31 can be designed to yield virtually any resistive force.

Extension versus force and contractive force can be determined by ASTM 882-02 with the following modifications. A sample representative of the extensible material disposed in the waist region should be collected for the test. In the test a 5.08 cm by 15.24 cm (2 inch by 6 inch) sample is cut from the material such that the edges are straight. The sample is clamped into the tensile tester. The clamps are attached 10.16 cm (4 in) from each other on the sample. The sample is pulled steadily at a speed of 2.54 cm/min (1 in/min) to 20% extension and then immediately returned to 0% (4 in. spacing between the clamps) at the same steady speed. Data, extension in mm and force in grams, should be collected at a rate of at least 1 data point per second. The data can be graphed to provide a curve of % extension versus force such that the extension at various tensile/contractive forces can be determined. The extension force can be determined by the extension curve and the contractive force can be determined by the return curve. This test should be repeated at 30, 40, 50, 60, 70, 80, 90 and 100% extension using a new specimen for each test. A representative sampling should be made for each condition.

To compare the extension force and contractive force of one pull-on diaper to another, the diaper in question is applied to a representative group of wearers within the specified size range of the diaper and the circumferential waist dimension of the diaper and/or wearer is determined. The circumferential waist dimension of the diaper as worn is then compared to the diaper waist circumference in a new unextended state. The % extension is derived by the following:

(As-worn waist circumference−original waist circumference)/original waist circumference)

Once the percentage waist extension is calculated, a correlating force can be established using the above-described method. It should thus be appreciated that, for a given diaper, a force-% extension relationship can be determined as described above.

Description of the Absorbent Assembly

As shown in FIGS. 1-5, the absorbent assembly 200 has left and right laterally opposing side flaps 247a and 247b. The side flaps can be formed by folding portions of the absorbent assembly toward the longitudinal axis 42, to form both the respective side flaps 247a and 247b and the side edges 237a and 237b of the absorbent assembly 200. Alternatively, the side flaps can be formed by attaching an additional layer or layers to the absorbent assembly 200 at or adjacent to each of the respective side edges 237a and 237b. In embodiments in which the side flaps are formed by attaching an additional layer or layers to the absorbent assembly, each of the additional layer or layers can be attached at or adjacent to its laterally distal edge.

In embodiments in which portions of the absorbent assembly 200 are folded laterally inward to form the side flaps 247a and 247b, the absorbent assembly 200 can simply be folded loosely or can be creased along a portion of each of its side edges 237a and 237b. For example, it may be desirable to form creases along portions of the side edges 237a and 237b in the crotch region 37 in order to impart a more finished appearance to the pull-on diaper 20. Alternatively or in addition to creasing, a portion of each of the folded side flaps 247a and 247b adjacent to the side edges 237a and 237b can be attached to the interior surface 202 of the absorbent assembly 200 to achieve a similar result.

The side flaps can overlap the absorbent core 250, i.e., the proximal edges 255a and 255b of the side flaps can lie laterally inward of the respective left side edge 257a and right side edge 257b of the absorbent core 250. Alternatively, the side flaps may not overlap the absorbent core. The side flaps preferably are water vapor-permeable, i.e., breathable, at least in the crotch region 37 where they form side barriers when the diaper is worn, as described in detail below.

In the exemplary pull-on diaper 20 shown in FIG. 1, the absorbent assembly 200 extends the full length of the backsheet strips 100a and 100b between the front waist edges 139a and 139b and the back waist edges 141a and 141b. Such a full length configuration may be desirable in order to minimize the amount of waste material and the difficulty associated with the manufacture of the pull-on diaper 20, especially when the method used to manufacture the pull-on diaper 20 requires the introduction of the material or materials for the absorbent assembly 200 in the form of a continuous web or multiple continuous webs. Also, such a full length configuration may be desirable in order to isolate the skin of the wearer from the backsheet strips. Alternatively, the absorbent assembly 200 can be shorter and extend less than the full length of the backsheet strips. Such a shorter configuration may be desirable in order to minimize the total amount of material used and the cost of the pull-on diaper 20.

Each of the side flaps 247a and 247b is attached to the interior surface 202 of the absorbent assembly 200 in attachment zones located at or adjacent to the front edge 236 and the back edge 238. For example, in the pull-on diaper 20 shown in FIG. 1, the left side flap 247a is attached to the interior surface 202 of the absorbent assembly 200 in attachment zones 251a and 251c, while the right side flap 247b is attached to the interior surface 202 in attachment zones 251b and 251d. The attachment zones may have equal areas or may be unequal in area.

Between the attachment zones, the proximal edges 255a and 255b of the side flaps 247a and 247b remain free, i.e., are not attached to the interior surface 202 of the absorbent assembly 200. Also between the attachment zones, each side flap preferably includes a longitudinally extensible flap elastic member that is attached adjacent to the proximal edge of the side flap. For example, in the pull-on diaper 20 shown in FIG. 1, elastic strands 267a and 267b are attached adjacent to the respective proximal edge 255a and 255b of the side flaps. The flap elastic member can be enclosed inside folded hems, such as the hems 271a and 271b shown in FIG. 4. Alternatively, the flap elastic member can be sandwiched between two layers of the absorbent assembly or may be attached on a surface of the absorbent assembly and remain exposed.

When stretched, the flap elastic members allow the proximal edges of the side flaps to extend to the flat uncontracted length of the absorbent assembly, as shown in FIG. 1. When allowed to relax, the flap elastic members contract to gather the portions of the proximal edges along which the flap elastic members are attached. For example, when the exemplary pull-on diaper 20 is in a relaxed condition, as shown in FIG. 11, the elastic strands 267a and 267b contract to gather the proximal edges 255a and 255b of the side flaps 247a and 247b. The contractive forces of the elastic strands pull the front waist region 36 and the back waist region 38 toward each other and thereby bend the absorbent assembly 200 and the entire pull-on diaper 20 into a “U” shape in which the interior of the “U” shape is formed by the interior portions of the diaper. Because the proximal edges remain free between the attachment zones, the contractive forces of the elastic strands lift the proximal edges 255a and 255b of the side flaps 247a and 247b away from the interior surface 202 of the absorbent assembly and thereby raise the side flaps into position to serve as side barriers. The lateral spacing of the lifted proximal edges is selected to allow the deposit of bodily wastes from the lower torso of the wearer into the space between the raised side flaps. The width of each of the side flaps 247a and 247b in effect becomes its height when the free portion of its proximal edge is lifted and the side flap is raised. This height preferably is selected to allow the lifted proximal edges 255a and 255b to fit into the leg creases of the body of the wearer to form seals to help prevent the leakage of deposited bodily waste out of the diaper.

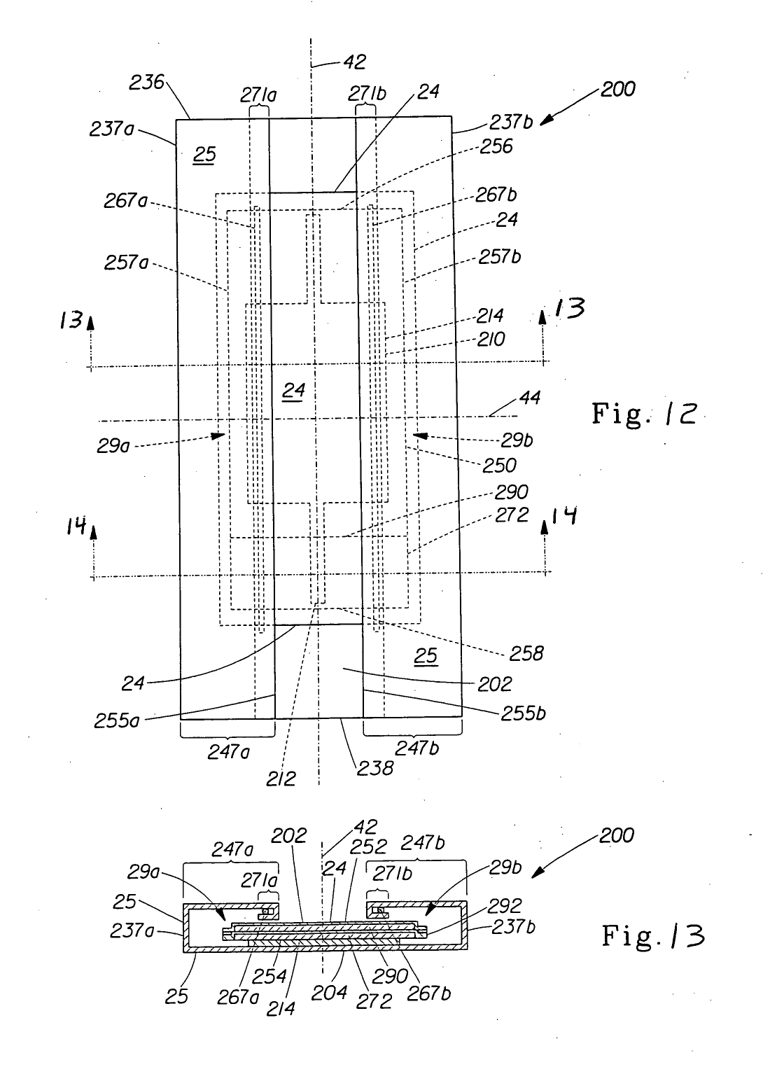

As shown in FIGS. 12-14, the absorbent assembly 200 includes an absorbent core 250 that serves to absorb and retain liquid bodily waste materials. The absorbent core 250 has a front edge 256, a back edge 258, a left side edge 257a, a right side edge 257b, an interior surface 252, and an exterior surface 254.

The absorbent assembly 200 can include an upper covering sheet that is disposed in a face-to-face arrangement with the interior surface 252 of the absorbent core 250 in addition to a lower covering sheet that is disposed in a face-to-face arrangement with the exterior surface 254 of the absorbent core 250 and the interior surfaces 102a and 102b of the respective backsheet strips 100a and 100b. If both are present, such an upper covering sheet and lower covering sheet can be attached together to contain the absorbent core 250 between them and thereby form the absorbent assembly 200. For example, in the exemplary absorbent assembly 200 shown in FIGS. 12-14, an upper covering sheet 24 and a lower covering sheet 25 are attached together at or adjacent to the side edges 237a and 237b of the absorbent assembly 200 in attachment zones 29a and 29b.

The upper covering sheet is water-permeable and allows liquid bodily waste to pass through its thickness to the absorbent core. The upper covering sheet preferably is formed of a soft material that will not irritate the skin of the wearer, for example a synthetic nonwoven such as spunbond or carded polyethylene, polypropylene, polyester, or rayon.

A portion or the whole of either or both of the upper covering sheet and the lower covering sheet can be water vapor-permeable, i.e., breathable.

Alternatively or in addition, the lower covering sheet can include a water-impermeable layer that is formed of a suitable material, for example a film of polyethylene or another polyolefin, a microporous breathable film, a hydrophobic nonwoven, or a film formed of coextruded layers of polyolefin layers. For example, a suitable coextruded film is available from Clopay Plastic Products Co. of Mason, Ohio, U.S.A. under the designation of M18-327. A multi-layer lower covering sheet, such as a laminate of a film and a nonwoven, can also be suitable and can be oriented with the nonwoven disposed exteriorly to provide the feel and appearance of a cloth-like outermost layer, with the nonwoven disposed interiorly to separate the film from the skin of the wearer, or with nonwovens disposed both exteriorly and interiorly.

The upper covering sheet and the lower covering sheet can extend to the same width and the same length. Alternatively, one or more of the edges of one of the covering sheets can lie distally relative to the respective edge or edges of the other covering sheet. For example, the upper covering sheet can extend longitudinally only to an extent sufficient to cover the absorbent core and to be attached to the lower covering sheet adjacent to either the front or the back edge of the absorbent core, while the lower covering sheet can extend longitudinally beyond the upper covering sheet toward or to the adjacent waist edges of the backsheet strips. Such a longitudinally extended lower covering sheet can serve to isolate the skin of the wearer from a portion of the backsheet strips as may be desirable, for example, when the diaper is worn under conditions in which contact between the skin and a backsheet film could be uncomfortable. Similarly, the upper covering sheet can extend laterally only to an extent sufficient to cover the absorbent core, or to an extent sufficient to be attached to the lower covering sheet adjacent to either the left or the right side edge of the absorbent core. The lower covering sheet can extend laterally beyond the upper covering sheet. For example, in the exemplary absorbent assembly 200 shown in FIG. 4, the upper covering sheet 24 extends laterally only a relatively small distance beyond the side edges 257a and 257b of the absorbent core 250 and is attached to the lower covering sheet 25 in this area. The lower covering sheet 25 in this exemplary absorbent assembly extends laterally beyond the upper covering sheet 24 and is folded to form the side flaps 247a and 247b.

The absorbent assembly and the backsheet strips can be attached together over any part or the whole of the length of the absorbent assembly. Preferably, the absorbent assembly is attached on its exterior surface to the backsheet strips in laterally opposing longitudinally extending attachment zones such as the exemplary attachment zones 220a and 220b shown in FIGS. 2-5, 12-14, and 16. The portions of the backsheet strips that lie outside such an attachment pattern are not restrained by attachment to the absorbent assembly and therefore can remain extensible. For example, a relatively narrow longitudinally extending attachment zone such as left attachment zone 220a leaves the majority of the width of the left backsheet strip 100a freely extensible and thereby allows extension of the left backsheet strip 100a in the lateral direction.

Within the extent of the attachment zones, the absorbent assembly can be attached to the backsheet strips continuously or intermittently. For example, a film of an adhesive can be applied continuously over the entire area of the attachment zones and then used to continuously attach the absorbent assembly to the backsheet strips. As an alternative example, an adhesive can be applied discontinuously at and inside the boundaries of the attachment zones, such as in the form of dots, stripes, beads, spirals, etc., and then used to attach the absorbent assembly to the backsheet strips.

The absorbent core can be attached to the lower covering sheet over any part or the whole of the area of the absorbent core. Preferably, the absorbent core is attached on its exterior surface to the lower covering sheet in a cruciform attachment pattern, i.e., in an attachment pattern that forms or is arranged in a cross or “+” shape. The cruciform attachment pattern can be contiguous, i.e., all of its portions can be touching or connected throughout the pattern in an unbroken sequence, or can include detached portions and thereby lack contiguity but still be arranged such that the shape of the overall pattern is a cruciform. An exemplary contiguous cruciform attachment pattern 210 is shown in FIGS. 12-14. When an adhesive is used for the attachment, less may be necessary in a cruciform attachment pattern than in a more extensive attachment pattern. In addition, the portions of the lower covering sheet that lie outside such a cruciform attachment pattern are not restrained by attachment to the absorbent core and therefore remain extensible. In particular, a relatively narrow longitudinally extending portion 212 of a cruciform attachment pattern 210 like that shown in FIGS. 12 and 14 leaves the majority of the width of the lower covering sheet 25 in the front waist region 36 and in the back waist region 38 freely extensible and thereby allows extension of the lower covering sheet 25 in the lateral direction in these regions. A relatively wide laterally extending portion 214 of a cruciform attachment pattern 210 like that shown in FIGS. 12-13 prevents the portion of the lower covering sheet 25 in the crotch region 37 to which the absorbent core 250 is attached from shifting relative to the absorbent core 250 in that region and thereby contributes to the effectiveness of the raised side flaps. For example, if the lower covering sheet in the crotch region 37 were free to shift laterally, the raised side flaps 247a and 247b might distort and fail to maintain contact with the body or become improperly positioned.

Within the extent of the cruciform attachment pattern, the absorbent core can be attached to the lower covering sheet continuously or intermittently. For example, a film of an adhesive can be applied continuously over the entire area of the cruciform attachment pattern and then used to continuously attach the absorbent core to the lower covering sheet. As an alternative example, an adhesive can be applied discontinuously at and inside the boundaries of the cruciform attachment pattern, such as in the form of dots, stripes, beads, spirals, etc., and then used to attach the absorbent core to the lower covering sheet.

When the pull-on diaper 20 is pulled onto the body of the wearer, a force will be applied by the diaper 20 to the waist region of the user to secure the diaper 20 onto the body of the wearer. Forces applied to the diaper 20 during application are simulated in FIG. 22 as opposing laterally outward forces F1 and F2 applied to the left and right side edges 137a and 137b, respectively, in the front waist region 36 and back waist region 38. Upon application of forces F1 and F2, the diaper 20 extends to a significantly greater degree in the waist region than in the crotch region thereby creating angled lines of tension 203. In embodiments where both the backsheet strips 100 and the absorbent assembly 200 are extensible, the lines of tension 203 are directed from portion 214 of the cruciform pattern 210 to the side edges 137a and 137b in both the front and back waist regions 36 and 38. The angled lines of tension 203 provide an internal support structure 201 integral with the absorbent assembly 200 and the backsheet strips 100, that receive forces from the core 250 and transmits the forces toward the waist regions of the pull-on diaper, specifically toward the closed side interfaces. It should be appreciated that the lines of tension 203, while still providing the internal support structure, would assume a different structural configurations if the absorbent assembly 200 was non-extensible, or if the if the backsheet strips 100 were non-extensible.

Suitable absorbent materials for the absorbent core 250 are well-known and can comprise any absorbent material that is generally compressible, conformable, non-irritating to the wearer's skin, and capable of absorbing and retaining liquids such as urine and other certain body exudates. The absorbent core 250 may comprise a wide variety of liquid-absorbent materials commonly used in disposable diapers and other absorbent articles such as comminuted wood pulp, which is generally referred to as air felt. Examples of other suitable absorbent materials include creped cellulose wadding; melt blown polymers, including co-form; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials. The absorbent core 250 can further comprise minor amounts (typically less than 10%) of non-liquid absorbent materials, such as adhesives, waxes, oils and the like. Exemplary absorbent structures for use as the absorbent assemblies are described in U.S. Pat. No. 4,610,678 (Weisman et al.); U.S. Pat. No. 4,834,735 (Alemany et al.); U.S. Pat. No. 4,888,231 (Angstadt); U.S. Pat. No. 5,260,345 (DesMarais et al.); U.S. Pat. No. 5,387,209 (Dyer et al.); U.S. Pat. No. 5,397,316 (LaVon et al.); U.S. Pat. No. 5,625,222 (DesMarais et al.). These absorbent materials can be used separately or in combination.

Many known absorbent materials can be used in a discrete form, i.e., in the form of fibers, granules, particles, and the like. Such a discrete form of an absorbent material can be immobilized by an adhesive that attaches the discrete pieces together to form a coherent layer or that attaches the discrete pieces to a substrate layer, such as a covering sheet, or that attaches the discrete pieces both to each other and to the substrate layer. Alternatively, the core 250 can comprise an absorbent polymer material in contact with a thermoplastic material. The absorbent polymer material can be further mixed with an absorbent fibrous material, such as airfelt material, or absorbent core 250 can be substantially airfelt free, as described in U.S. patent application Ser. No. 10/776,851 (Becker et al), published as U.S. Publication. No. 2004/0162536.

The absorbent core 250 includes a storage component 272 that serves to absorb and retain liquid bodily waste materials. Suitable known materials for the absorbent core storage component include cellulose fibers in the form of comminuted wood pulp, which is commonly known as “airfelt”, layers or sheets of a natural or synthetic fibrous material or materials, a superabsorbent polymer or polymers, etc. These absorbent materials can be used separately or in combination. Many known absorbent materials can be used in a discrete form, i.e., in the form of fibers, granules, particles, and the like. Such a discrete form of an absorbent material can be immobilized by an adhesive that attaches the discrete pieces together to form a coherent layer or that attaches the discrete pieces to a substrate layer or that attaches the discrete pieces both to each other and to the substrate layer.

The absorbent core 250 can include an acquisition component 290 in addition to one or more storage components. The absorbent core acquisition component serves to acquire deposited liquid bodily waste material and transfer it to the absorbent core storage component. Any porous absorbent material which will imbibe and partition liquid bodily waste material to the storage component or components 272 can be used to form the acquisition component 290. Preferred materials for the acquisition component 290 include synthetic fiber materials, open celled polymeric foam materials, fibrous nonwoven materials, cellulosic nonwoven materials, and various combination synthetic/cellulosic nonwoven materials. For example, the acquisition component can be formed of a nonwoven web or webs of synthetic fibers including polyester, polypropylene, and/or polyethylene, natural fibers including cotton and/or cellulose, blends of such fibers, or any equivalent materials or combinations of materials. Examples of such acquisition materials are more fully described in U.S. Pat. No. 4,950,264 issued to Osborn on Aug. 21, 1990. High loft nonwoven acquisition materials suitable for the acquisition component of the present invention can be obtained from Polymer Group, Inc., (PGI), 450 N.E. Blvd, Landisville, N.J. 08326, U.S.A., under the material code designation of 98920.

Such an absorbent core acquisition component 290 is shown overlying the absorbent core storage component 272 in FIGS. 12-14. A separation sheet 292 of, e.g., a tissue or a nonwoven material, can be disposed between the absorbent core storage component 272 and the absorbent core acquisition component 290 to help ensure that none of the gel formed by a superabsorbent polymer reaches the skin of the wearer. This separation sheet 292 can extend laterally beyond the side edges 257a and 257b of the absorbent core 250 and the upper covering sheet 24 can be attached to the separation sheet 292. In this arrangement, the liquid bodily waste material that is deposited onto the upper covering sheet 24 will pass through the thickness of the upper covering sheet 24 to be absorbed by the absorbent core acquisition component 290, and some or all of it can then pass through the thickness of the separation sheet 292 and then be absorbed and retained by the absorbent core storage component 272.

As shown in FIG. 15, in some exemplary embodiments, an absorbent core storage component 272 can include the discrete form of an absorbent material that is immobilized in pockets formed by a layer of a thermoplastic material, such as a hot melt adhesive, that intermittently contacts and adheres to a substrate sheet, while diverging away from the substrate sheet at the pockets. Absorbent core components having such structures and being suitable for the storage of liquid bodily wastes are described in co-pending and commonly assigned U.S. patent applications Ser. Nos. 10/776,839 and 10/776,851, both filed on 11 Feb. 2004 in the name of Ehrnsperger et al. An exemplary absorbent core storage component 272 having such a structure is shown in FIG. 15. In this absorbent core storage component 272, particles 270 of a superabsorbent polymer are contained inside pockets 280 formed by a layer 275 of a thermoplastic material. The absorbent core storage component can include both particles of a superabsorbent polymer and airfelt and both materials can be contained inside the pockets formed by the layer of the thermoplastic material. Alternatively, as shown in FIG. 15, an exemplary absorbent core storage component can contain no airfelt and therefore the component can be made relatively thinner and more flexible for the comfort of the wearer. In addition, the particles of the superabsorbent polymer can be immobilized relatively more easily in the absence of airfelt. As shown in FIG. 15, the layer 275 of the thermoplastic material intermittently contacts and adheres to a substrate sheet 274 at the areas of attachment 282. Between the areas of attachment 282, the layer 275 diverges away from the substrate sheet 274 to form the pockets 280. The layer 275 can have the form of a sheet of fibers of the thermoplastic material through which the liquid bodily waste can pass to be absorbed by the particles 270 of the superabsorbent polymer.

In FIG. 15, a separate thermoplastic layer covering sheet 276 is shown overlying the layer 275 of the thermoplastic material. Alternatively, the separate thermoplastic layer covering sheet 276 can be omitted. As another alternative, two absorbent core storage components each like that shown in FIG. 15 except for the omission of the thermoplastic layer covering sheet 276 can be superposed with one absorbent core storage component inverted such that the respective substrate sheets distally oppose each other. In such a combination of absorbent core storage components, either or both of the distally opposing substrate sheets can serve respectively as either or both of an upper covering sheet and a lower covering sheet for the absorbent assembly. Alternatively, the absorbent assembly can include a separate lower covering sheet that is disposed between the absorbent core and the interior surface of the backsheet and/or a separate upper covering sheet that is disposed interiorly of the absorbent core.

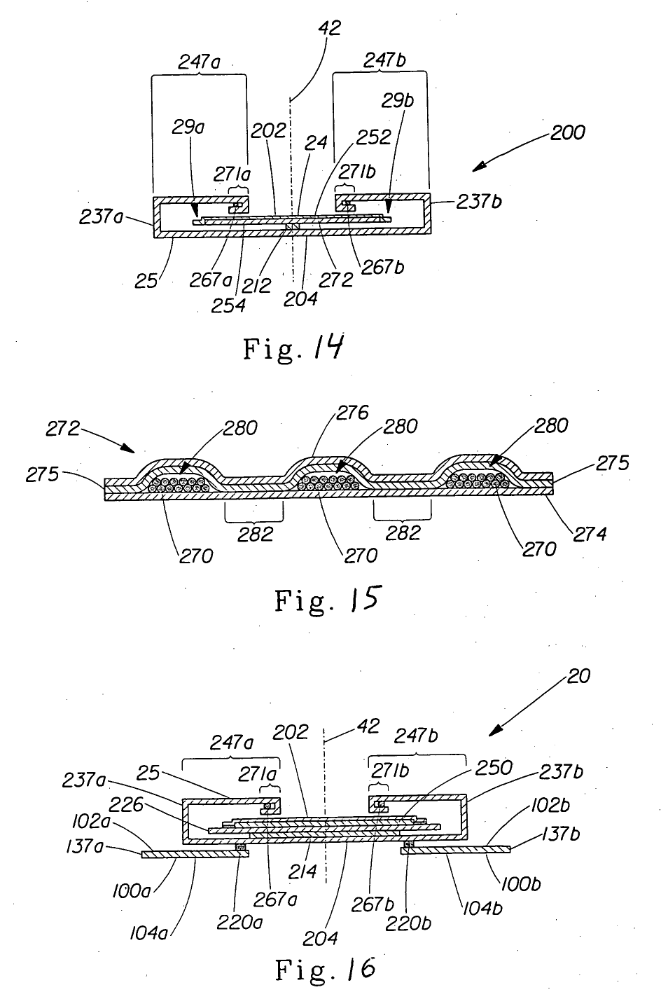

The absorbent assembly can include an additional bottom sheet of a film or other water-impermeable material to enhance the protection against leakage. For example, as shown in FIG. 16, an additional bottom sheet 226 of a film or other water-impermeable material can be attached inside the absorbent assembly between the lower covering sheet 25 and the absorbent core 250. Alternatively, the additional bottom sheet can be attached to the absorbent assembly exteriorly of the lower covering sheet. This additional bottom sheet can extend laterally less far than either or both of the left side edge 237a and the right side edge 237b of the absorbent assembly 200, as shown in FIG. 16, or can extend laterally to overlap one or both of the side edges of the absorbent assembly.

When such an additional bottom sheet is attached inside the absorbent assembly between the lower covering sheet and the absorbent core, the additional bottom sheet can be attached to the lower covering sheet in a cruciform attachment pattern similar to that shown in FIG. 12, thus leaving the portions of the lower covering sheet that lie outside the cruciform attachment pattern unrestrained by attachment to the additional bottom sheet and allowing these portions to be extensible. For example, a laterally extending portion 214 of such a cruciform attachment pattern is shown in FIG. 16.

Alternatively or in addition, the additional bottom sheet in such an embodiment can be attached in such a cruciform attachment pattern to the absorbent core, thus leaving the portions of the additional bottom sheet that lie outside the cruciform attachment pattern unrestrained by attachment to the absorbent core and therefore allowing these portions to be extensible. In such an embodiment, even if the additional bottom sheet is attached to the lower covering sheet in a pattern other than a cruciform, the lower covering sheet is not indirectly restrained by the absorbent core and therefore is allowed to be extensible.

Description of Shape of Article

The finished diaper can have a generally rectangular shape, as in the exemplary pull-on diaper 20 shown in FIGS. 1 and 2. Such a generally rectangular configuration can be desirable in order to minimize the amount of waste material and the difficulty associated with the manufacture of the pull-on diaper 20. Alternatively, the diaper can have side edges 137a and 137b that are not straight, but instead are curved and/or notched, thereby giving an overall shape in plan view of an hourglass or of an “I” to the pull-on diaper 20. Such a non-rectangular configuration may be desirable in order to impart a tailored appearance to the pull-on diaper 20 when it is worn. Such a non-rectangular configuration may also be desirable in order to impart an impression that the pull-on diaper 20 will fit comfortably between the legs of a wearer.

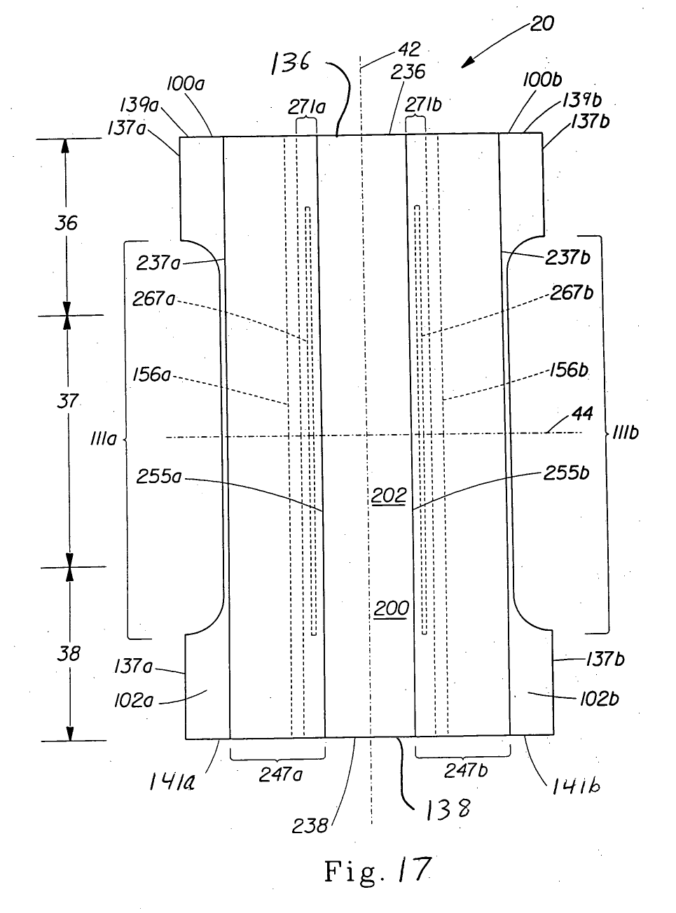

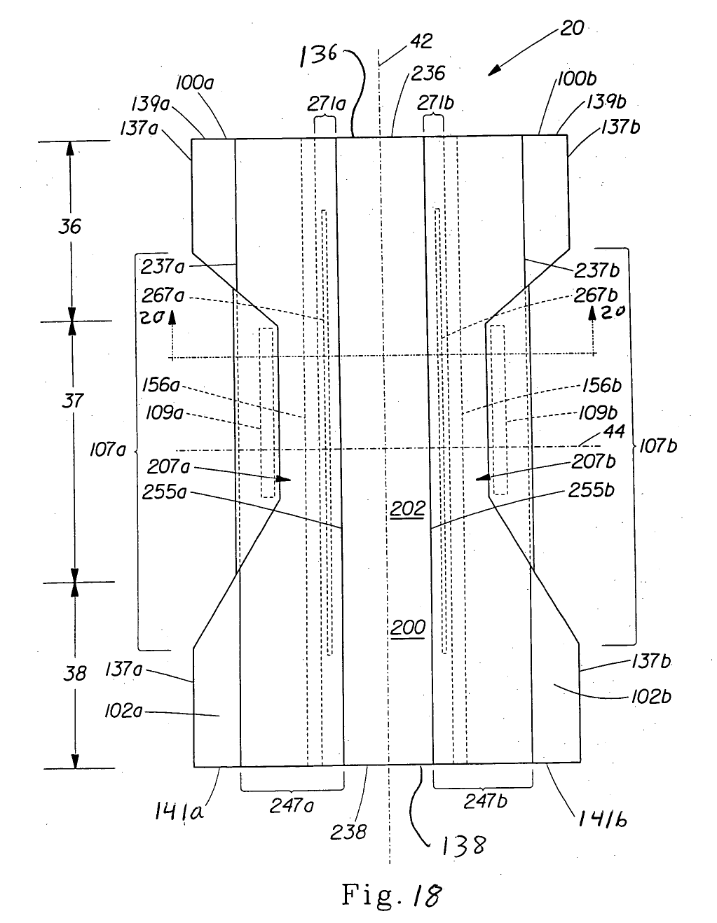

A non-rectangular configuration of the diaper can be formed in any one of several ways. For example, laterally distal portions can be removed from the diaper 20 to make its lateral dimension at and adjacent to the lateral axis 44 smaller than its lateral dimension at and adjacent to the front waist edge 136 and smaller than its lateral dimension at and adjacent to the back waist edge 138, i.e., to make the diaper narrower in the crotch region 37 than at the waist edges. An exemplary form of such a non-rectangular configuration of the diaper is shown in FIG. 17. As shown in this figure, portions of the backsheet strips 100a and 100b can be removed to form laterally opposing side notches 111a and 111b, while leaving the backsheet strips longitudinally continuous.

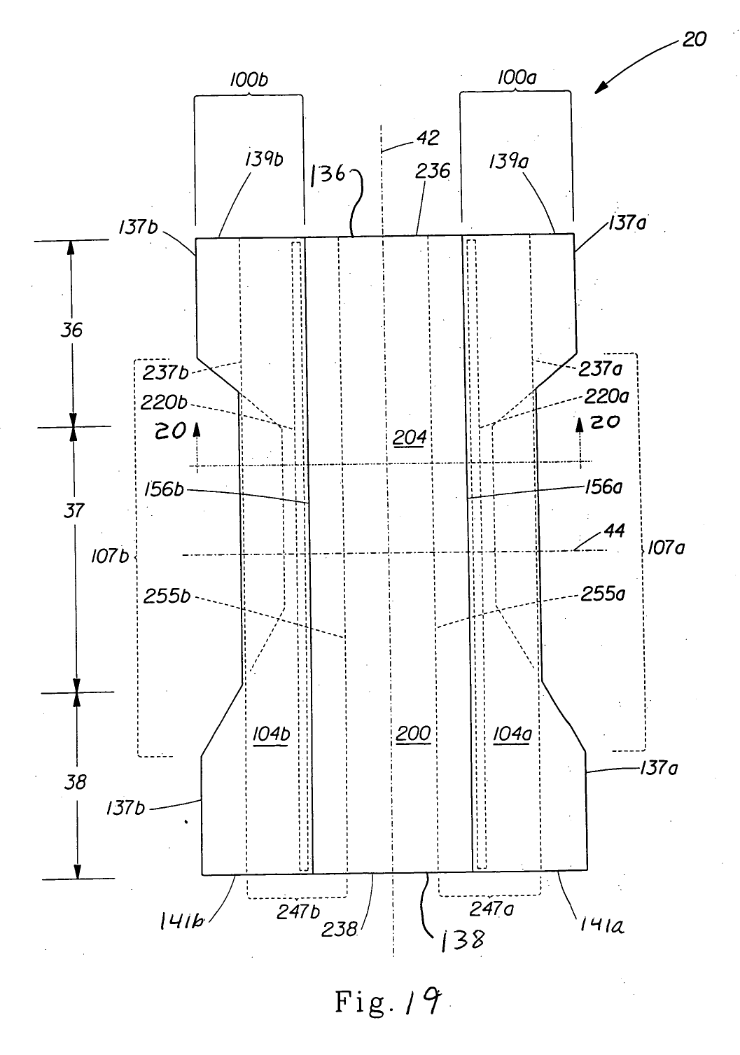

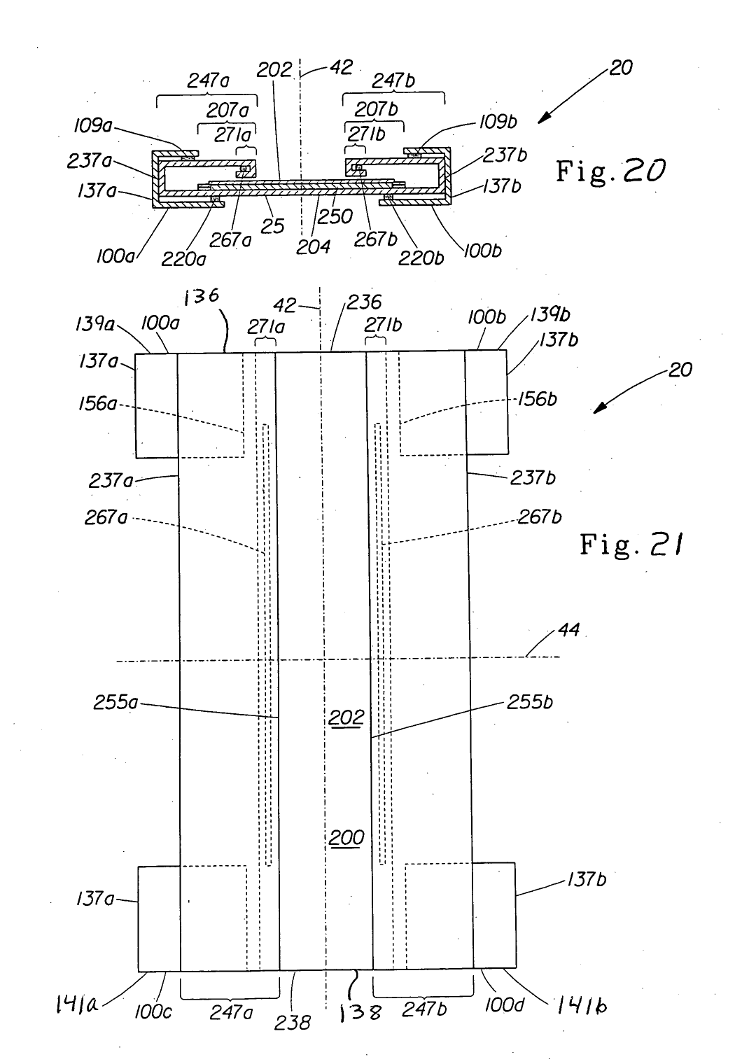

Alternatively, a portion of each of the side edges 137a and 137b can be folded laterally inward in order to form a non-rectangular configuration of the diaper. For example, as shown in FIGS. 18-20, laterally opposing portions 107a and 107b of the backsheet strips 100a and 100b in the crotch region 37 can be folded laterally inward to overlap the respective side flaps 247a and 247b and can be attached to the side flaps, for example, in the respective attachment zones 109a and 109b. Preferably, each of the folded laterally opposing portions 107a and 107b extends laterally only a part of the way from the respective side edge 237a or 237b of the absorbent assembly 200 toward the longitudinal axis 42, thus leaving uncovered respective exposed portions 207a and 207b of the side flaps, which form side barriers when the diaper is worn, as described above, particularly when the side barriers are breathable.

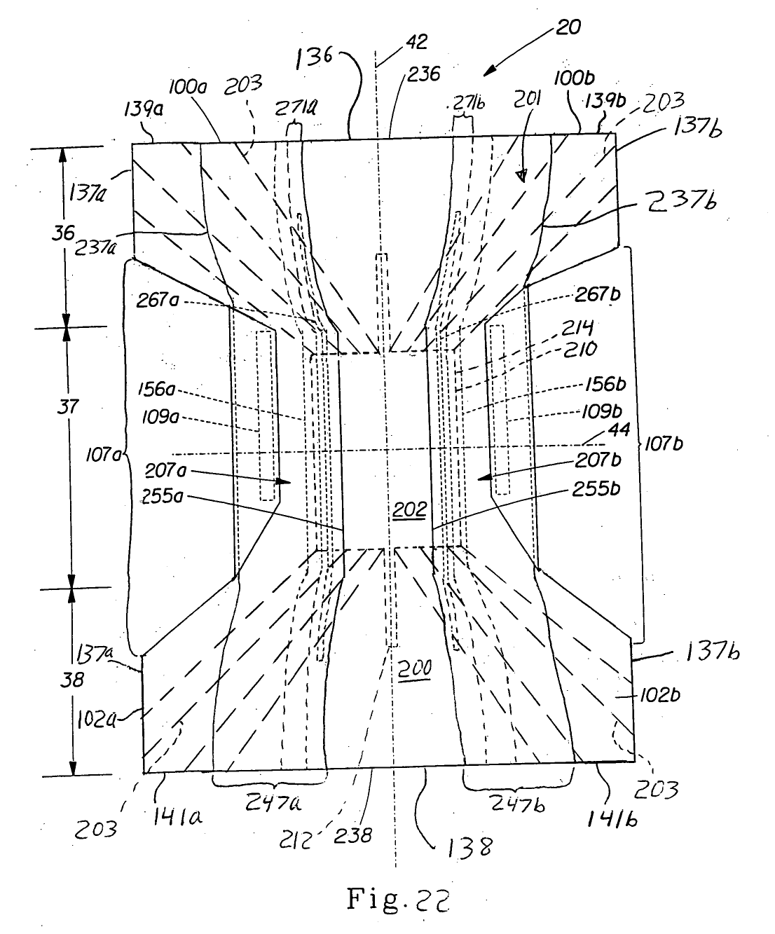

As another alternative, the backsheet strips 100a and 100b can be made longitudinally discontinuous in order to form a non-rectangular configuration of the diaper. For example, as shown in FIG. 21, the backsheet strips can be separated into a left front backsheet strip 100a, a left back backsheet strip 100c, a right front backsheet strip 100b, and a right back backsheet strip 100d, and each of these backsheet strips can extend laterally beyond the side edges 237a and 237b of the absorbent assembly 200, thereby imparting an “I” shape to the pull-on diaper 20.

All documents cited in the Detailed Description of the Invention are, in relevant part, incorporated herein by reference, however the citation of any document is not construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments and/or individual features of the present invention have been illustrated and described, it would be obvious to those skilled in the art that various other changes and modifications can be made without departing from the spirit and scope of the invention. Further, it should be apparent that all combinations of such embodiments and features are possible and can result in preferred executions of the invention. Therefore, the following claims are intended to cover all such changes and modifications that are within the scope of this invention.

What is claimed is:

1. A disposable pant-like garment having a front waist region, a back waist region, and a crotch region disposed between the waist regions, the garment comprising:
laterally opposing first and second side edge regions, each side edge region including a pre-closed side interface, wherein the pre-closed side interfaces define an encircled waist opening and a pair of encircled leg openings;
an absorbent assembly having an interior surface and an exterior surface and laterally opposing longitudinally extending side flaps, each side flap having longitudinally opposing ends and a longitudinally extending proximal edge, each side flap being attached adjacent to its ends to the interior surface of the absorbent assembly and having a longitudinally extending elastic gathering member attached adjacent to its proximal edge such that when allowed to relax, the elastic gathering member contracts and lifts the proximal edge away from the interior surface of the absorbent assembly, thereby raising the side flap to form a side barrier; and
a pair of laterally opposing longitudinally extending backsheet strips attached to the exterior surface of the absorbent assembly,
wherein at least a portion of the absorbent assembly is laterally extensible.

2. The disposable pant-like garment as recited in claim 1 wherein the extensible portion comprises a web material including at least two distinct laterally extending altered regions each containing a pattern of generally longitudinally oriented alternating ridges and valleys created by a deformation of the web material and also containing an unaltered region located between the altered regions, such that the deformed web material can be laterally extended to a given extent with the application of relatively less force than that required to laterally extend the same web material to the same given extent before the deformation.

3. The disposable pant-like garment as recited in claim 1 wherein at least a portion of the absorbent assembly between the backsheet strips is laterally extensible.

4. The disposable pant-like garment as recited in claim 1 wherein at least a portion of at least one of the backsheet strips is laterally extensible.

5. The disposable pant-like garment as recited in claim 4, wherein both backsheet strips are laterally extensible.

6. The disposable pant-like garment as recited in claim 1 wherein at least a portion of one of the waist regions is laterally extensible to a greater degree than at least a portion of the crotch region.

7. The disposable pant-like garment as recited in claim 1 wherein the absorbent assembly includes an absorbent core and a lower covering sheet at least a portion of which is disposed exteriorly of the absorbent core and laterally opposing portions of the lower covering sheet are folded laterally inward to form the respective side flaps.

8. The disposable pant-like garment as recited in claim 7 wherein the lower covering sheet is water-impermeable.

9. The disposable pant-like garment as recited in claim 7 wherein the absorbent assembly further comprises a water-impermeable bottom sheet at least a portion of which is disposed between the lower covering sheet and the absorbent core.

10. The disposable pant-like garment as recited in claim 1 wherein the backsheet strips are attached to the absorbent assembly in laterally opposing longitudinally extending attachment zones.

11. The disposable pant-like garment as recited in claim 1 wherein each of the backsheet strips comprises a water-impermeable layer.

12. The disposable pant-like garment as recited in claim 1 wherein the absorbent assembly comprises an absorbent core storage component.

13. The disposable pant-like garment as recited in claim 12 wherein the absorbent core storage component contains no airfelt.

14. The disposable pant-like garment as recited in claim 12 wherein the absorbent assembly comprises an absorbent core acquisition component.

15. The disposable pant-like garment as recited in claim 1, wherein the first and second interfaces comprise a permanent closure member.

16. The disposable pant-like garment as recited in claim 1, wherein the first and second interfaces comprise a refastenable closure member.

17. The disposable pant-like garment as recited in claim 1, further comprising an elastic waist member disposed in at least one of the front waist region and back waist region, the waist member being configured to provide a contractive force onto a wearer after the garment has been applied to the wearer.

18. The disposable pant-like garment as recited in claim 17, wherein the elastic member comprises a laterally extending strip at least partially overlying the absorbent assembly.

19. The disposable pant-like garment as recited in claim 17, wherein the elastic member extends from and between the closed side interfaces.

20. A disposable pant-like garment having a front waist region, a back waist region, and a crotch region disposed between the waist regions, the garment comprising:
laterally opposing first and second side edge regions, each side edge region including a pre-closed side interface, wherein the pre-closed side interfaces define an encircled waist opening and a pair of encircled leg openings;
an absorbent assembly having an interior surface and an exterior surface and laterally opposing longitudinally extending side flaps, each side flap having longitudinally opposing ends and a longitudinally extending proximal edge, each side flap being attached adjacent to its ends to the interior surface of the absorbent assembly and having a longitudinally extending elastic gathering member attached adjacent to its proximal edge such that when allowed to relax, the elastic gathering member contracts and lifts the proximal edge away from the interior surface of the absorbent assembly, thereby raising the side flap to form a side barrier; and
a pair of laterally opposing longitudinally extending backsheet strips attached to the exterior surface of the absorbent assembly,
wherein at least a portion of at least one of the backsheet strips is laterally extensible.

21. The disposable pant-like garment as recited in claim 20, wherein the first and second interfaces comprise a permanent closure member;

22. The disposable pant-like garment as recited in claim 20, wherein the first and second interfaces comprise a refastenable closure member.

23. The disposable pant-like garment as recited in claim 20, wherein at least a portion of both of the backsheet strips is laterally extensible.

24. The disposable pant-like garment as recited in claim 20, further comprising an elastic waist member disposed in at least one of the front waist region and back waist region, the waist member being configured to provide a contractive force onto a wearer after the garment has been applied to the wearer.

25. The disposable pant-like garment as recited in claim 24, wherein the elastic waist member comprises a laterally elongated strip at least partially overlying the at least one of the backsheet strips.

26. The disposable pant-like garment as recited in claim 24, wherein the elastic waist member extends from and between the closed side interfaces.

27. A disposable pant-like garment having a front waist region, a back waist region, and a crotch region disposed between the waist regions, the garment comprising:
laterally opposing first and second side edge regions, each side edge region including a pre-closed side interface, wherein the pre-closed side interfaces define an encircled waist opening and a pair of encircled leg openings;
an absorbent assembly having an interior surface and an exterior surface and laterally opposing longitudinally extending side flaps, each side flap having longitudinally opposing ends and a longitudinally extending proximal edge, each side flap being attached adjacent to its ends to the interior surface of the absorbent assembly and having a longitudinally extending elastic gathering member attached adjacent to its proximal edge such that when allowed to relax, the elastic gathering member contracts and lifts the proximal edge away from the interior surface of the absorbent assembly, thereby raising the side flap to form a side barrier; and
a pair of laterally opposing longitudinally extending backsheet strips attached to the exterior surface of the absorbent assembly,
wherein the absorbent assembly comprises an absorbent core storage component containing no airfelt.
